(19) United States
(12) Patent Application Publication
(10) Pub. No.: US 2026/0221281 A1
(43) Pub. Date: Jul. 30, 2026

(54) PREDICTING PATIENT OUTCOMES RELATED TO PANCREATIC CANCER

(71) Applicant: Valar Labs, Inc., Palo Alto, CA (US)

(72) Inventors: Anirudh Rajiv JOSHI, Palo Alto, CA (US); Damir Vrabac, Palo Alto, CA (US); Viswesh Krishna, Cupertino, CA (US); Vrishab Krishna, Palo Alto, CA (US); Vivek Uday Nimgaonkar, Baltimore, MD (US); Ekin Tiu, Palo Alto, CA (US)

(21) Appl. No.: 19/147,678

(22) PCT Filed: Nov. 9, 2023
(86) PCT No.: PCT/US2023/079259

§ 371 Date: Jul. 11, 2025

Related U.S. Application Data
(60) Provisional application No. 63/439,061, filed on Jan. 13, 2023.

(51) Int. Cl. G16H 50/20; G16H 50/70

(52) U.S. Cl. CPC G16H 50/20; G16H 50/70

(57) ABSTRACT

Disclosed are systems and methods for an artificial intelligence based pathology platform that can provide prognostic value to clinicians. For example, the platform can predict outcomes related to a pancreatic cancer, and may include the steps of obtaining a histological sample of a pancreatic cancer tumor of a patient, determining a feature set for the histological sample by applying a deep learning module trained on a population of histological samples of cancer tumors of the same type as the obtained histological sample of the cancer tumor, and generating an outcome set for the patient by applying a second model to the determined feature set. Additional methods for identifying a signature for a histological sample that is indicative of treatment outcome are disclosed.

### CROSS-REFERENCE TO RELATED APPLICATIONS

This application claims the benefit of priority from U.S. Provisional Application No. 63/439,061, filed on Jan. 13, 2023, and entitled, “PREDICTING PATIENT OUTCOMES RELATED TO PANCREATIC CANCER,” the contents of which are hereby fully incorporated by reference.

### TECHNICAL FIELD

The present disclosure relates to predicting patient outcomes related to pancreatic cancer, e.g., using machine learning models.

### BACKGROUND

Cancer is a leading cause of death worldwide, and accounts for close to one in six deaths. However, many cancers can be cured if treated effectively and early. Pathological samples from a cancer patient are often analyzed for clinical metrics of risk and may be used to determine the most appropriate treatment for that patient. However, conventional methods that estimate clinical metrics of risk are often limited in design and may overestimate or underestimate the risk of progression, recurrence and/or treatment failure of cancer within a patient.

### SUMMARY

Embodiments of the present disclosure include techniques for applying a machine learning model to pathology samples to predict outcomes, stratify risk, and predict responses to cancer therapies, and particularly, pancreatic cancer therapies.

In some embodiments, a method performed by at least one processor for predicting outcomes related to pancreatic cancer, may include the steps of obtaining a histological sample of a pancreatic cancer tumor of a patient, determining a feature set for the histological sample by applying a deep learning module trained on a population of histological samples of cancer tumors of the same type as the obtained histological sample of the cancer tumor, and generating an outcome set for the patient by applying a second model to the determined feature set. Optionally, the outcome set may include at least one of a risk category, or risk-score for at least one of time on treatment, time to treatment discontinuation, recurrence free survival, progression free survival, event free survival, overall survival, response to therapy, or disease-free survival. The cancer may be pancreatic ductal adenocarcinoma including unresectable pancreatic ductal adenocarcinoma, metastatic pancreatic ductal adenocarcinoma, and resectable pancreatic adenocarcinoma. The method may also include the step of providing a set of recommended therapies responsive to the determined feature set for the histological sample. Optionally, the feature set for the histological sample may include at least one of morphology data, tissue region data, spatial relationship data, colocalization data, and hotspot data. In some embodiments, the deep learning module includes a U-Net model, where the U-Net model comprises a fully convolutional neural network having an encoder and decoder. In some embodiments, the deep learning module is trained on the population of histological samples of cancer tumors of the same type as the obtained histological sample of the cancer tumor to determine nuclei location and shape data. In some embodiments, determining a feature set for the histological sample further includes the steps of determining locations of tissue within the histological sample, detecting positions of nuclei and cells of interest within the determined locations of tissue, determining at least one of morphologic, geometric, and textural features for each of the detected nuclei and cells of interest, and determining a spatial location feature for each of the detected nuclei and cells of interest. Optionally, the second model may include a multivariate model. In some embodiments, the multivariate model includes a Cox proportional hazards (CPH) model. In some embodiments, training the second model on non-histological data includes at least one of medical images, clinical variables, genomics, and medical text. Further, the second model may be trained to determine a signature, wherein the signature comprises the combination of histological features and weights. In some embodiments, the method includes the step of administering to the patient the particular treatment type, responsive to the outcome set for a particular treatment type. Further, the method may also include the step of displaying, on a graphical user interface, at least a portion of the outcome set.

In some embodiments, a non-transitory computer-readable medium may store instructions that, when executed on one or more processors, cause the one or more processors to obtain a histological sample of a pancreatic cancer tumor of a patient, determine a feature set for the histological sample by applying a deep learning module trained on a population of histological samples of cancer tumors of the same type as the obtained histological sample of the cancer tumor, and generate an outcome set for the patient by applying a second model to the determined feature set. Optionally, the instructions may also cause the one or more processors to display, on a graphical user interface, at least a portion of the outcome set. Optionally, the instructions may also cause the one or more processors to determine the feature set for the histological sample by determining locations of tissue within the histological sample, detecting positions of nuclei and cells of interest within the determined locations of tissue, determining at least one of morphologic, geometric, and textural features for each of the detected nuclei and cells of interest, or determining a spatial location feature for each of the detected nuclei and cells of interest. In some embodiments the second model includes a multivariate model.

In some embodiments, a system for predicting outcomes related to a pancreatic cancer, includes at least one server communicatively coupled to a user device by a network, wherein the at least one server further comprises a non-transitory memory storing computer-readable instructions and at least one processor. The execution of the computer-readable instructions causing the at least one server to train a deep learning module on a population of histological samples of pancreatic cancer tumors, wherein the deep learning module comprises a U-net model, train a second model on feature set data and outcomes data, wherein the second model comprises a multivariate model, obtain a histological sample of a cancer tumor of a patient, wherein the cancer tumor is of the same type as the population of histological samples of cancer tumors, determine a feature set for the histological sample by applying the trained deep learning module, and generate an outcome set for the patient by applying the trained multivariate model to the determined feature set.

In some embodiments, the feature set includes at least one of morphology data, tissue region data, spatial relationship data, colocalization data, and hotspot data. Optionally, determining the feature set may include the execution of computer-readable instructions causing the at least one server to: determine locations of tissue within the histological sample, detect positions of nuclei and cells of interest within the determined locations of tissue, determine at least one of morphologic, geometric, and textural features for each of the detected nuclei and cells of interest, or determine a spatial location feature for each of the detected nuclei and cells of interest. In some embodiments, the outcome set includes at least one of a risk category, or risk-score for at least one of time on treatment, time to treatment discontinuation, recurrence free survival, progression free survival, event free survival, overall survival, response to therapy, or disease-free survival. In some embodiments, a graphical user interface may be communicatively coupled to the at least one server and configured to display a portion of the outcome set.

In some embodiments, a method for providing a set of recommended therapies related to pancreatic cancer includes the steps of obtaining a histological sample of a pancreatic cancer tumor of a patient, determining a feature set for the histological sample by applying a deep learning module trained on a training data set, where the training data set comprises pancreatic cancer treatment outcomes for a prospective therapy, generating an outcome set for the patient by applying a second model to the determined feature set, determining a signature for the histological sample by thresholding the generated outcome set, and providing a set of recommended therapies related to pancreatic cancer responsive to the determined signature. Optionally, the prospective therapy may be a combination therapy. In some embodiments, the outcome set comprises at least one of a risk category, or risk-score for at least one of time to treatment discontinuation, time on treatment, recurrence free survival, progression free survival, event free survival, overall survival, response to therapy, or disease-free survival. Pancreatic cancer may be at least one of unresectable pancreatic ductal adenocarcinoma, metastatic pancreatic ductal adenocarcinoma and resectable pancreatic ductal adenocarcinoma. Optionally, the feature set for the histological sample may include at least one of morphology data, tissue region data, spatial relationship data, colocalization data, and hotspot data. Optionally, the deep learning module may include a U-Net model, where the U-Net model comprises a fully convolutional neural network having an encoder and decoder. In some embodiments, the deep learning module may be trained on the population of histological samples of cancer tumors of the same type as the obtained histological sample of the cancer tumor to determine nuclei location and shape data. In some embodiments determining a feature set for the histological sample further includes determining locations of tissue within the histological sample, detecting positions of nuclei and cells of interest within the determined locations of tissue, determining at least one of morphologic, geometric, and textural features for each of the detected nuclei and cells of interest, and determining a spatial location feature for each of the detected nuclei and cells of interest. Optionally, the second model includes a multivariate model. The multivariate model may be a Cox proportional hazards (CPH) model. In some embodiments, the second model may be trained on non-histological data comprising at least one of medical images, clinical variables, genomics, and medical text. In some embodiments the signature includes the combination of histological features and weights. In some embodiments, the method includes the additional step of administering to the patient the particular treatment type, responsive to the outcome set for a particular treatment type. Optionally, the method may include the step of displaying, on a graphical user interface, at least a portion of the outcome set.

### BRIEF DESCRIPTION OF THE DRAWINGS

The embodiments described herein will more fully understood from the following detailed description taken in conjunction with the accompanying drawings. The drawings are not intended to be drawn to scale. For the purposes of clarity, not every component may be labeled in every drawing. In the drawings:

FIG. 1 is a block diagram for a system for an artificial intelligence based pathology platform in accordance with some embodiments of the present disclosure;

FIG. 2 is a flow chart for a method for an artificial intelligence based pathology platform in accordance with some embodiments of the present disclosure;

FIG. 3 is a block diagram for a computer system for an artificial intelligence based pathology platform in accordance with some embodiments of the present disclosure;

FIG. 4 is a diagram for a system for an artificial intelligence based pathology platform in accordance with some embodiments of the present disclosure;

FIG. 5 is a diagram for experimental results for an artificial intelligence based pathology platform in accordance with some embodiments of the present disclosure;

FIG. 6 is a schematic for an experiment utilizing an artificial intelligence based pathology platform in accordance with some embodiments of the present disclosure;

FIG. 7 is a diagram for experimental results for an artificial intelligence based pathology platform in accordance with some embodiments of the present disclosure;

FIG. 8 is a diagram for experimental results for an artificial intelligence based pathology platform in accordance with some embodiments of the present disclosure;

FIG. 9 is a schematic for an experiment utilizing an artificial intelligence based pathology platform in accordance with some embodiments of the present disclosure;

FIG. 10 is a schematic for an experiment utilizing an artificial intelligence based pathology platform in accordance with some embodiments of the present disclosure;

FIG. 11 is a schematic for an experiment utilizing an artificial intelligence based pathology platform in accordance with some embodiments of the present disclosure; and

FIGS. 12A-20 are diagrams for experimental results for an artificial intelligence based pathology platform in accordance with some embodiments of the present disclosure.

### DETAILED DESCRIPTION

Embodiments of the present disclosure are directed towards systems and methods for predicting outcomes related to cancers. Examples of outcomes include time on treatment, time to treatment discontinuation, recurrence free survival, progression free survival, event free survival, overall survival, disease-free survival. In some embodiments, a deep learning module is trained on a collection of histological data received from a population of patients having cancer tumors as well as their response to treatments and recurrence of cancer rates.

As one example, the disclosed systems and methods may be applied to patients having pancreatic ductal adenocarcinoma who may be treated with chemotherapies like combination drugs such as Gemcitabine and Nab-Paclitaxel (i.e., Gem+nabPTX) or FOLFIRINOX (i.e., folinic acid, fluorouracil, irinotecan, and oxaliplatin combination). For example, the disclosed systems and methods may be utilized to develop pancreatic cancer treatment plans by determining the appropriate and most beneficial first line chemotherapy treatment for a particular patient. Although applications related to pancreatic cancer are discussed herein, it is envisioned that applications related to other cancers may utilize similar approaches to those described herein.

The trained deep learning module can be applied to histological samples from a cancer tumor of a patient in order to predict survival or recurrence or response to therapy for a given treatment or therapy. For example, the deep learning module can be applied to a histological sample for a cancer tumor for a patient, in order to determine a feature set for the histological sample.

In some examples, the deep learning module includes one or more processes for determining the feature set. For example, the deep learning module first determines locations of tissue within the histological sample using threshold based techniques. After detecting areas of tissue cells, the deep learning module applies a U-net architecture to detect positions of nuclei and cells of interest within the identified locations of tissue. In some examples, the U-net architecture is composed of a plurality of convolutional layers configured to first distinguish between objects and background, determine segments of interest within an image based on determined boundaries of the objects, and classify objects. In some embodiments, U-Net model comprises a fully convolutional neural network having an encoder and decoder. The deep learning module may also determine morphologic, geometric, and textural features for each of the detected nuclei and cells of interest. For example, the nuclei may be classified into classes such as Neoplastic, Connective, No-Neoplastic/Epithelial, Necrotic, and Inflammatory. Additionally, the deep learning module may be configured to determine a spatial location feature for each of the detected nuclei and cells of interest based on their correlation and/or overlap. Once a feature set is obtained, the disclosed systems and methods may generate an outcome set for a patient by applying a second multivariate model (e.g., Cox proportional hazards model). The outcome set may be used to predict the likelihood of recurrence/progression/survival/treatment response of cancer in the patient and guide treatment choices by clinicians. The outcomes set can be a risk score on the outcomes or risk categories like high/low risk of 12 month survival.

For example, the artificial intelligence based pathology platform described herein may provide clinicians with an adjunctive tool that classifies a patient population into “high-risk”/“positive” or “low-risk”/“negative” for one or more possible outcomes. This classification may occur at the time of disease diagnosis based on an analysis of a histological sample. Further, the disclosed artificial intelligence based pathology platform may leverage existing workflow and standards of care by using histological samples and other data that is routinely collected and provide clinicians with prognostic information regarding risk of treatment failure and/or likelihood of survival and/or recurrence for a cancer for a given therapy prior to the initiation of a particular therapy.

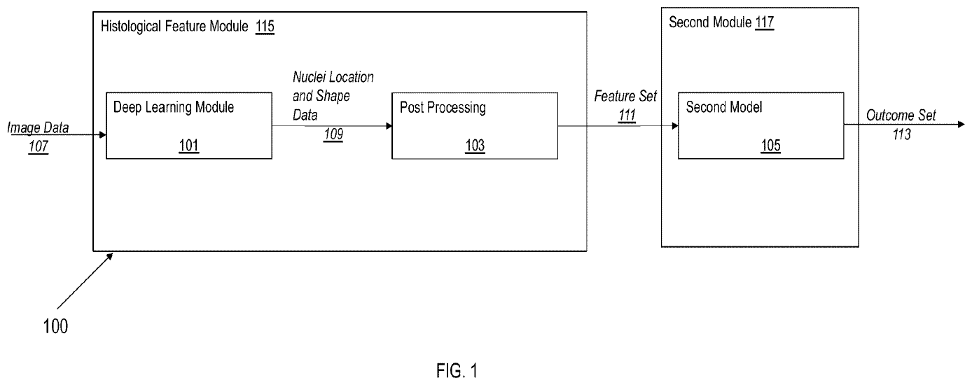

FIG. 1 shows a block diagram for an example of an artificial intelligence based pathology platform 100 for predicting patient outcomes related to cancer. The platform 100 includes a histological feature module 115 that includes a deep learning module 101 and one or more post processing algorithms 103. The platform 100 also includes a second module 117 which includes one or more algorithmic models. For example, a second model 105 may be included in the second module 117. Image data 107 is provided to a deep learning module 101 that is configured to output nuclei location and shape data 109. In some embodiments, post processing algorithms 103 are applied to the nuclei location and shape data 109 in order to generate a feature set 111. The feature set 111 are input into a second model 105 that produces an outcome set 113.

Image data 107 may include digital pathology slides of cancer specimens. For example, this may include whole slide images (WSI) or virtual microscopy images which are digital scans of samples (e.g., tissue sections). WSI or virtual microscopy images may allow for the digitalization of glass slide images. In some embodiments, the image data 107 may be stained using hematoxylin and eosin stains (H&E stains). In some embodiments, the image data 107 may include a 256×256 input image of a histopathology slide. In some embodiments, the image data 107 may be stained using immunohistochemistry (IHC) techniques.

In some examples, the image data 107 corresponds to slides taken in connection with pancreatic ductal adenocarcinoma.

In some embodiments the deep learning module 101 includes a nuclei segmentation and classification algorithm. In some embodiments, the deep learning module 101 utilizes CellCS. In some embodiments, the deep learning module 101 utilizes a U-Net architecture. The nuclei segmentation and classification approach (CellCS) may form the deep learning model that uses the U-Net architecture as its basic element.

The deep learning module 101 may be configured to distinguish between objects of interest and background, perform segmentation, and classify identified nuclei into respective cell classes. For example, the deep learning module 101 may include three independent convolutional layers of size 3×3×128 that are applied to the output of the final layer of the U-Net model to respectively predict pixel-level (i) normalized object instance probabilities to distinguish between objects of interest and the background, (ii) 32-ray radial distances to boundaries of objects for segmentation, and (iii) cell class probabilities for the classification of nuclei into any number of cell classes.

The first of the three independent convolutional layers may form the object instance layer, configured to help distinguish between objects of interest and background. For a given input, the object instance layer may predict normalized scores for each pixel in the input region to identify if that pixel is associated with a nuclei region or the background.

The second of the three independent convolutional layers may form the segmentation layer, which is configured to determine boundaries of objects for segmentation. In particular, for each pixel in some embodiments 32 radial distance values are predicted to identify the edge of the predicted segmentation for a pixel if that pixel was part of an object.

The third of the three independent convolutional layers may form the cell classification layer. The cell classification layer may compute cell class probabilities corresponding to the likelihood a given cell is of a particular cell class. In some embodiments, the cell classification layer is composed of an n+1 channel predicted mask where a single channel mask corresponds to normalized predictions for each pixel corresponding to a certain cell class on the patch. The classes consist of n cell classes and a background class.

Examples of cell classes may include 5, 14, or any other number of different classes. In some embodiments, the five classes may include Neoplastic, Connective, No-Neoplastic/Epithelial, Necrotic or Inflammatory.

In some embodiments, the deep learning module 101 may refine predictions of a single object across multiple pixels using non-maximal suppression above a given object threshold. The majority class probability prediction across the entire object is used to classify the object. For a given segmentation mask, a set of all segmentation masks that were suppressed using non-maximal suppression are used to refine the pixel level segmentation of the object.

In some embodiments, the segments may undergo a shape refinement procedure applied by the deep learning module 101. In a shape refinement procedure, all polygons for an object instance are rasterized as binary masks and aggregated by majority vote in order to obtain the mask of an object instance.

The deep learning module 101 may be evaluated and validated on the basis of its model loss. In some embodiments, model loss is composed of three separate components: a distance regression component, a probability map component and a classification component. The separate components may be aggregated with a weighted sum to form the complete model loss. For example, the distance regression component may correspond to the clipped absolute difference between the 32 distance predictions at each pixel locations. The clipped absolute difference may be weighted by the corresponding ground truth probability for that pixel location and the resulting tensor is then average pooled and normalized by the mean value of the ground truth probability map to produce a scalar corresponding to the distance regression loss.

The probability map component may correspond to the average pooled binary cross-entropy between the predicted and ground truth probability map.

The classification component may correspond to the average pooled cross entropy between the predicted probability of each type per pixel with the class-map.

Together, the final model loss for the deep learning model can be characterized as follows:

Model Loss={Weight Factor}×{Distance Regression}+{Probability Map Component}+{Classification Component}

In some examples, the ground truths include an instance mask with a class map connecting instance indices to class indices. The Euclidean Distance Transform is applied to a binarized mask of nucleus or background pixel level classifications to generate a ground truth probability map. The 32 radial distances to the boundaries of objects are generated at each pixel location to create the distance map. A class map image is generated with each pixel equal to the class index if it is part of a nucleus or zero if it is not.

In some embodiments, the deep learning module 101 is trained using training data that includes annotations of cell segmentation and classification data that is annotated from patches extracted from histopathology images and slides. The training data may include marked cell centroids for all cells in the patches along with the classification of the cells in the region. The cell classes may include 5, 14, or any number of different classes. In some embodiments, the five classes may include Neoplastic, Connective, No-Neoplastic/Epithelial, Necrotic or Inflammatory.

In some embodiments, the deep learning module 101 applies tissue segmentation, nuclei segmentation and geometric feature extraction to the image data 107. In some embodiment, the deep learning module 101 may receive image data 107 that is preprocessed. Examples of pre-processing of the image data 107 include excluding background regions of a whole slide image. In some embodiments, excluding background regions may involve applying color-based thresholding using the lightness channel of the CIELAB color space that was binarized using Otsu's method. For example, in some embodiments, image data 107 may be preprocessed using a single intensity threshold to separate pixels within the received image data 107 into foreground or background. Further, in some embodiments, pre-processing may include identifying patches of appropriate size that would be provided to the deep learning module 101. For example, in some embodiments patches of size 2132×2132 (533×533 μm) are extracted from tissue regions. Pre-processing may also include one or more processes for detecting and removing artifacts.

As discussed above, the deep learning module 101 may then be used to segment and classify each nucleus automatically into a class (e.g., Neoplastic, Connective, No-Neoplastic/Epithelial, Necrotic, Inflammatory). Further, the deep learning module 101 may perform geometric feature extraction on the resulting classified nuclei. For example, the centroids, bounding boxes, and contours of the nuclei may be calculated. The geometric feature extraction may result in shape data.

In some examples, the deep learning module 101 is configured to output nuclei location and shape data 109. In some embodiments the histological feature module 115 may include one or more computer vision techniques to provide nuclei location and shape data 109.

A post processing algorithm 103 may be applied to the output of deep learning module 101 in order to generate a feature set 111. The feature set 111 may include morphology data, tissue region data, spatial relationship data, colocalization data, and hotspot data. The feature set 111 may be computed from the geometric features extracted from the classified nuclei. For example, the feature set 111 may be computed from centroids and contours for each nuclei.

The feature set 111 may include morphology data. Morphology data may be descriptive of morphometric features of nuclei. For example, morphology data may include information about the dimensions, perimeter, area, curvature and eccentricity of nuclei. Morphology data may be computed using segmentation masks, and provide characterizations of the area surrounding nuclei. For example, the morphology data may indicate areas of neoplastic nuclei.

The feature set 111 may include tissue region data. Tissue region data may classify tissue regions according to the maximum cell type proportion predicted by the deep learning module 101. For example, the centroids of nuclei determined by geometrical extraction by the deep learning module 101 may be used to create a spatial mesh using the Delaunay triangulation algorithm. Measurements of the area and perimeter of the triangles formed by the mesh may then be calculated. Examples of features that provide tissue region data include features related to identifying an area of tumor, an area of stroma, and the density of tumor.

The feature set 111 may also include spatial relationship data. The spatial relationship data may indicate relationships between nuclei and cells. For example, spatial relationship data may include spatial statistics of nucleus centroids, nucleus features, and triangle features of different types which may be calculated globally at a slide level and locally in sub-regions (variable sized regions in the slide). Examples of the spatial relationship data may also include colocalization metrics, spatial correlations between features, Moran's indices, measures of spatial entropy, and total variance.

One example of spatial relationship data is colocalization data. Colocalization may be computed on regions within the whole slide image. In some embodiments, colocalization data may be indicative of correlations of the counts of cells between multiple cell types. For example, the counts of cells may be computed on regions within the regions (sub-regions). The counts of cells in the sub-regions may be correlated across the region to compute the colocalization of the corresponding region. In some embodiments, the correlation may be computed by two different metrics: Pearson's correlation coefficient (PCC) and Mander's overlap coefficient (MOC). The sub-regions and the regions can be variable sized sections. Examples of spatial relationship data that may be included in a feature set include data regarding the colocalization of neoplastic and immune cells.

Another example of spatial relationship data is hotspot data. In some embodiments, a spatially connected set of regions within a whole slide image may be defined as a super-region. A super-region that meets a certain pre-defined specification may be considered a hotspot. For example, hotspot features may include the shapes, sizes, counts, and areas of the hotspot. In another example, hotspot data may reveal whether the number of nuclei in a particular super-region having a Neoplastic shape exceeds a threshold amount.

In some embodiments, one or more sub-features may be generated for each sub-region in a whole slide image. Sub-features may correspond to, but are not limited to, the shape and size of a cell, hues of a group of pixels, and the like. Features may be computed as an aggregation of sub-features, such that the features correspond to regions in the whole slide image. Each region may be composed of a set of sub-regions. Similarly, a super-region may be a collection or set of regions and a corresponding super-feature can be computed based on the features of the regions within the super-region. In some embodiments, hotspots may be defined as a super-region whose super-feature meets a defined threshold. A hotspot feature may be a geometrical feature of the hotspot itself.

In some embodiments, morphology data, tissue region data, spatial relationship data, colocalization data, and hotspot data may be aggregated across the whole slide image. For example, each of the morphology data, tissue region data, spatial relationship data, colocalization data and hotspot data may be computed for each sub-region within a whole slide image and then aggregated across the whole slide image with measures like mean, median, standard deviation, interquartile ranges and multiple percentile values (e.g., 5, 10, 15, 25 . . . 75, 85, 95, 99). Examples of features that may be aggregated include data indicating a 95th percentile neoplastic nuclear area.

In some embodiments, morphology data, tissue region data, spatial relationship data, colocalization data, and hotspot data may be aggregated to produce a final feature vector for the whole slide image.

The post processing 103 module of the histological feature module 115 may include one or more algorithms configured to intake the nuclei location and shape data 109 and output a feature set 111, including the morphology data, tissue region data, spatial relationship data, colocalization data, and hotspot data.

Algorithms included in the post processing 103 module may include those configured for determining morphologic, geometric, textural features of nuclei and/or cells. These may include algorithms for fitting ellipses, bounding boxes, algorithms for calculating the area of morphologic features, algorithms for calculating the hue and staining features. In some embodiments, additional artificial intelligence based models that are trained to calculate features within defined regions using supervised or unsupervised learning may be used.

The feature set may be input into a second module 117 which may include one or more additional algorithmic models. For example, the second module 117 may include second model 105 that is configured to produce an outcome set 113. In some embodiments the second model 105 may include a multivariate model. In some embodiments the second model 105 may include a Cox proportional hazards (CPH) model.

In some embodiments, the second module 117 may sub-select features to form a feature set that is strongly associated with the outcome of interest in the population. In order to do so, the histologic assay may be normalized on the training dataset by subtracting the mean and dividing by the standard deviation and then applying this transformation onto the test dataset. Features can then be pruned by training independent univariate cox proportional hazards models on the training set. Features that have a high concordance index on the training set may then be selected. In order to prevent overfitting towards any specific dataset, features that are associated with histopathological features that have been previously identified in the clinical literature may be subselected. For example features associated with stromal and neoplastic cell morphology were associated with differential therapy response.

In some embodiments the second model 105 may include a multivariate cox proportional hazards model with the sub selected features associated with the outcome of interest being trained on the entire training set. The second model 105 may be trained on a feature set based on a population of histological samples and known outcomes.

After training, the module may generate weights which will determine how features from the feature set are to be combined. These combination of weights and associated features may create a signature. By applying the signature to incoming feature set, the model may generate a risk category or risk score. The risk score or category can then predict an outcome such as recurrence or progression. For example, in some embodiments, a percentile threshold may be used to categorize “low” and “high” risk categories. For example, the 50th percentile response of the predicted expected lifetimes of data points in the training set was used to set the cut-off threshold between “low” and “high” risk categories.

In some embodiments, the second model 105 may also be trained on non-histological data including at least one of medical images, clinical variables, genomics, and medical text.

In some embodiments the second model 105 may generate an outcome set that includes at least one of a risk category, or risk-score for at least one of recurrence free survival, progression free survival, event free survival, overall survival, response to therapy, or disease-free survival. Recurrence free survival may refer to the length of time after primary treatment for a cancer ends that the patient survives without any signs or symptoms of that cancer. Progression free survival may refer to the length of time during and after the treatment of a disease, such as cancer, that a patient lives with the disease but it does not get worse. Event free survival may refer to the length of time after primary treatment for a cancer ends that the patient remains free of certain complications or events that the treatment was intended to prevent or delay. Overall survival may refer to the percentage of people in a study or treatment group who are still alive for a certain period of time after they were diagnosed with or started treatment for a disease, such as cancer. Response to therapy may respond to clinical observations such as tumor shrinkage, tumor death, and the like. Disease-free survival may refer to the length of time after primary treatment for a cancer ends that the patient survives without any signs or symptoms of that cancer.

Based on the outcome set, in some embodiments, the artificial intelligence platform may be configured to produce a graphical user interface for a clinician, printed reports, an indication for an electronic health record, and the like. In some embodiments, a clinician may be able to decide on a course of treatment based on the outcome set. In some embodiments, the platform 100 may be further configured to generate and provide a set of recommended therapies responsive to the determined feature set for the histological sample. For example, the platform 100 may be trained with histological samples and outcome data for a plurality of treatment options and provide recommendations for selecting a treatment option based on the histological features of a sample. In some embodiments, a graphical user interface may display at least a portion of the outcome set.

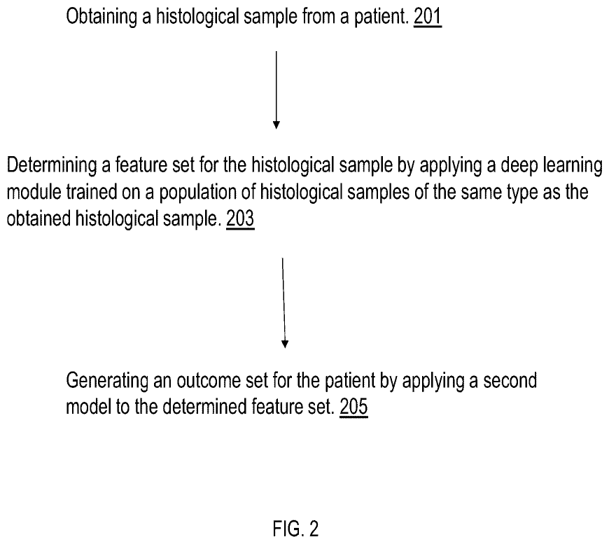

FIG. 2 illustrates a flowchart for a method built in accordance with some embodiments of the present disclosure. A method for predicting outcomes related to a cancer may include the step of obtaining a histological sample of a cancer tumor of a patient 201. In a second step, the method may determine a feature set for the histological sample by applying a deep learning module trained on a population of histological samples of cancer tumors of the same type as the obtained histological sample of the cancer tumor 203. In a third step, a method may generate an outcome set for the patient by applying a second model to the determined feature set 205.

As discussed above, the method may utilize components of the artificial intelligence pathology platform illustrated in FIG. 1. Accordingly, the method may also include training the deep learning module 101 on a population of histological samples of cancer tumors of the same type as the obtained histological sample of the cancer tumor to determine nuclei location and shape data 109.

Further, the method may include determining a feature set for the histological sample by determining locations of tissue within the histological sample, detecting positions of nuclei and cells of interest within the determined locations of tissue, determining at least one of morphologic, geometric, and textural features for each of the detected nuclei and cells of interest, and determining a spatial location feature for each of the detected nuclei and cells of interest.

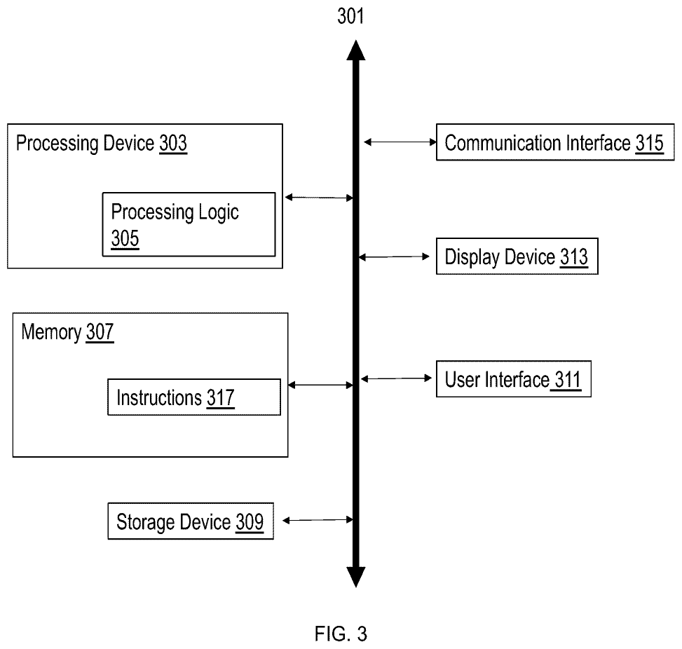

FIG. 3 illustrates a functional block diagram of a machine in the example form of computer system 300, within which a set of instructions for causing the machine to perform any one or more of the methodologies, processes or functions discussed herein may be executed. In some examples, the machine may be connected (e.g., networked) to other machines as described above. The machine may operate in the capacity of a server or a client machine in a client-server network environment, or as a peer machine in a peer-to-peer (or distributed) network environment. The machine may be any special-purpose machine capable of executing a set of instructions (sequential or otherwise) that specify actions to be taken by that machine for performing the functions describe herein. Further, while only a single machine is illustrated, the term “machine” shall also be taken to include any collection of machines that individually or jointly execute a set (or multiple sets) of instructions to perform any one or more of the methodologies discussed herein. In some examples, the platform 100 of FIG. 1 may be implemented by the example machine shown in FIG. 3 (or a combination of two or more of such machines).

Example computer system 300 may include processing device 303, memory 307, data storage device 309 and communication interface 315, which may communicate with each other via data and control bus 301. In some examples, computer system 300 may also include display device 313 and/or user interface 311. In some embodiments, the user interface 311 may include a graphical user interface.

Processing device 303 may include, without being limited to, a microprocessor, a central processing unit, an application specific integrated circuit (ASIC), a field programmable gate array (FPGA), a digital signal processor (DSP) and/or a network processor. Processing device 301 may be configured to execute processing logic 305 for performing the operations described herein. In general, processing device 303 may include any suitable special-purpose processing device specially programmed with processing logic 305 to perform the operations described herein.

Memory 307 may include, for example, without being limited to, at least one of a read-only memory (ROM), a random access memory (RAM), a flash memory, a dynamic RAM (DRAM) and a static RAM (SRAM), storing computer-readable instructions 317 executable by processing device 303. In general, memory 307 may include any suitable non-transitory computer readable storage medium storing computer-readable instructions 317 executable by processing device 303 for performing the operations described herein. Although one memory device 307 is illustrated in FIG. 3, in some examples, computer system 300 may include two or more memory devices (e.g., dynamic memory and static memory).

Computer system 300 may include communication interface device 315, for direct communication with other computers (including wired and/or wireless communication), and/or for communication with a network. In some examples, computer system 300 may include display device 313 (e.g., a liquid crystal display (LCD), a touch sensitive display, etc.). In some examples, computer system 300 may include user interface 311 (e.g., an alphanumeric input device, a cursor control device, etc.).

In some examples, computer system 300 may include data storage device 309 storing instructions (e.g., software) for performing any one or more of the functions described herein. Data storage device 309 may include any suitable non-transitory computer-readable storage medium, including, without being limited to, solid-state memories, optical media and magnetic media.

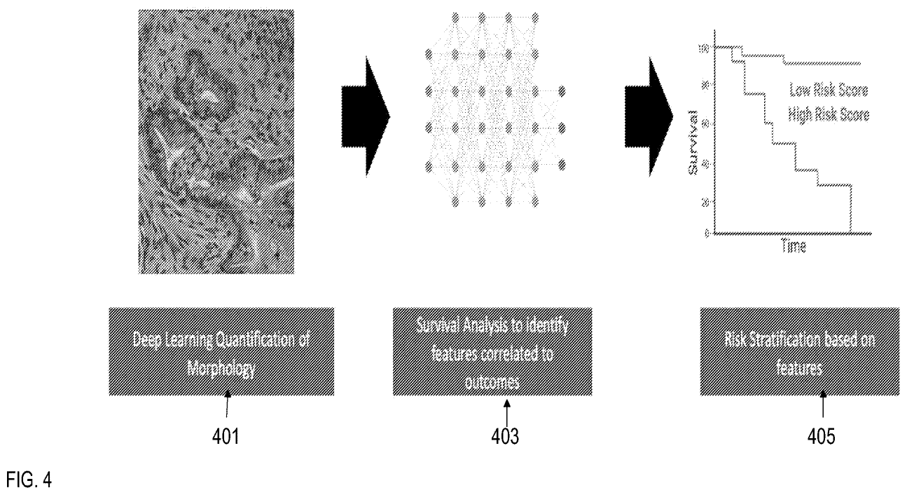

FIG. 4 is a diagram for a system for an artificial intelligence based pathology platform in accordance with some embodiments of the present disclosure. As illustrated in FIG. 4 and discussed herein, in a first step the artificial intelligence based pathology platform may apply deep learning to quantify morphology 401. Then in a second step, the pathology platform may apply survival analysis to identify features correlated to outcomes 403. And finally, in a third step, the pathology platform may apply risk stratification to identify patients having high or low risk scores based on the identified features 405.

In some embodiments, the system such as the one illustrated in FIG. 4 can be used to provide a set of recommended therapies to a patient with cancer, including pancreatic cancer. In such an embodiment, an artificial intelligence based pathology platform may obtain a histological sample of a pancreatic cancer tumor of a patient, determine a feature set for the histological sample by applying a deep learning module trained on a training data set, generate an outcome set for the patient by applying a second model to the determined feature set, determine a signature for the histological sample by thresholding the generated outcome set, and provide a set of recommended therapies related to pancreatic cancer responsive to the determined signature. Thresholding the generated outcome set may include determining the percentile of the predicted expected lifetimes of data points in the training set to set the cut-off threshold between “negative” and “positive” risk categories. For example, the pathology platform may be trained on clinical data including histological data as well as medical information regarding pancreatic cancer treatment outcomes for prospective therapies. In some embodiments, the prospective therapies may include combination therapies. The determined signature may be configured to provide predictive outcomes for any number of treatments affiliated with the combination therapy.

### Example 1: Application of Artificial Intelligence Based Platform to Determine First-Line Treatment Selection for Pancreatic Ductal Adenocarcinoma (PDAC)

### Overview

Patients diagnosed with metastatic pancreatic ductal adenocarcinoma (mPDAC) typically have poor prognoses, with patients having a median survival time of 10-12 months in metastatic cases. Physicians routinely administer first-line treatments such as FOLFIRINOX (FFX) and Gemcitabine+NAB-Paclitaxel (GNP) to patients with mPDAC. However, the methodology by which physicians determine which first-line treatment to apply to given patient is largely influenced by performance status, with fit patients more often receiving FOLFIRINOX (FFX) than Gemcitabine+Nab-Paclitaxel (GNP). Although the two first-line treatments may provide improved outcomes over gemcitabine monotherapy, no biomarkers routinely used in clinical practice can predict which therapy is optimal to facilitate a precision medicine approach. Accordingly, the disclosed artificial intelligence platform may be utilized to determine which first-line treatment is most appropriate for a given patient.

For example, an artificial intelligence platform built in accordance with the description herein was used to analyze digitized whole slide image (WSI) histologic sections derived from pre-treatment core biopsy specimens to stratify treatment outcomes for patients treated with two separate treatment regimens. A first treatment regimen involved a FOLFIRINOX (FFX) backbone. A second treatment regimen involved a Gemcitabine (GNP) backbone. The association of the histological assay stratification to disease specific survival (DSS), at multiple institutions were evaluated for each treatment regimen.

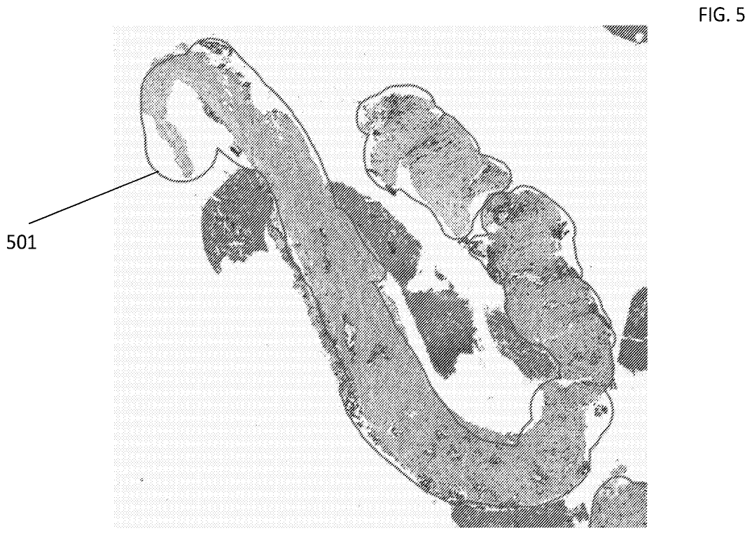

For example, FIG. 5 illustrates a whole slide image of a histologic section from a mPDAC cancer patient for which the artificial intelligence platform described herein determined improved disease specific survival under a Gemcitabine treatment over a FOLFIRINOX treatment regimen. Histological features 501 of the slide were determined by the artificial intelligence based platform.

### Data Set for AI Platform

A data set including digitized histological H&E sections corresponding to 145 metastatic PDAC patients treated with either first-line FFX or GNP was used to train an artificial intelligence platform such as the histological feature module 115 of FIG. 1. The data set was obtained from a retrospective study of mPDAC patients treated at two institutions (X and Y) from 2014 to 2021.

The full data set of mPDAC patients was separated into training and testing data sets for development and validation of histological feature module, such as histological feature module 115 of FIG. 1. For example, independent randomized training and test datasets were constructed for each treatment regime: FFX-treated (training set: 41 patients, testing set: 25 patients) and GNP-treated (training set: 49 patients, testing set: 30 patients).

### Model Training

A deep-learning algorithm analogous to the one contained in deep learning module 101 of FIG. 1 segmented nuclei to extract quantitative histological features. The extracted quantitative histological features were analogous to feature set 111 of FIG. 1.

Features associated with disease-specific survival (DSS) for the two treatment regimes (i.e., FFX and GNP) were identified utilizing univariate Cox proportional hazards (CPH) models for the respective training sets. The CPH model was analogous to the second model 105 of FIG. 1. The CPH model constructed V-FFX and V-GNP signatures. In other words, two signatures (i.e., V-FFX and V-GNP) corresponding to treatment by a FOLFIRINOX regimen and a Gemcitabine+NAB-Paclitaxel regimen were constructed. In this manner, signatures corresponding to treatment outcomes associated with each first-line regimen were determined. DSS stratification of the V-FFX and V-GNP signatures were examined using Kaplan-Meier analysis and the log-rank test and DSS percentages at twelve months were calculated on the respective test sets. Signatures may be indicative of histological features present within the sample image that are indicative of whether a particular treatment is better suited for the type of cancer associated with the sample.

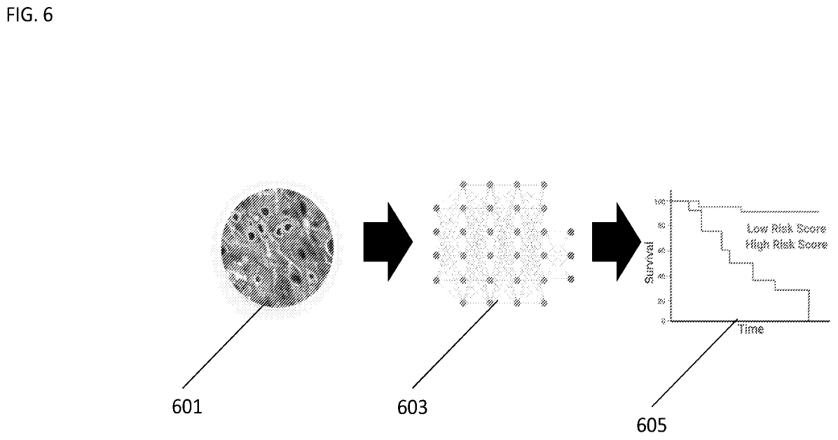

For example, FIG. 6 shows an overview of how the artificial intelligence based platform is used. In step 601, a deep learning model (analogous to first model 101 of FIG. 1) may be applied to image data. In a second step 603, a second model (analogous to model 105 of FIG. 1) may perform survival analysis on the data set. In a third step 605, the platform may predict the outcomes for patients to which a given image data sample belongs if they were treated under FFX or GNP, based at least in part on how closely related the received image data is to the V-FFX and V-GNP signature.

A second model, analogous to model 105 of FIG. 1, was trained on feature sets and identified outcomes from the data. The 50th percentile response of the predicted expected lifetimes of data points in the training set was used to set the cut-off threshold between “negative” and “positive” risk categories. Outcome stratification of the model was examined using Kaplan-Meier analysis, log-rank test and c-index on the test set. Overall survival rate was compared across negative and positive risk categories generated by the risk assessment model.

### Application of the Models

Scanned histologic images were analyzed through an imaging pipeline that included tissue segmentation, nuclei segmentation, and finally geometric feature extraction. Tissue was segmented via color-based thresholding to remove empty regions of the slide. Patches of size 2132×2132 were extracted from tissue regions and a validated deep learning model was used to segment and classify each nucleus automatically into five classes (i.e., Neoplastic, Connective, No-Neoplastic/Epithelial, Necrotic, Inflammatory). Descriptive morphometric features were then computed for each nucleus. Geometric features were then aggregated first at the patch, and subsequently at the patient level using summary statistics including the mean, standard deviation, skewness and kurtosis to produce the final feature vector for a patient. This feature vector was used as the input to a cox proportional hazards model that used the least absolute shrinkage and selection operator to identify the most correlated features with DSS along with their coefficients on the training set.

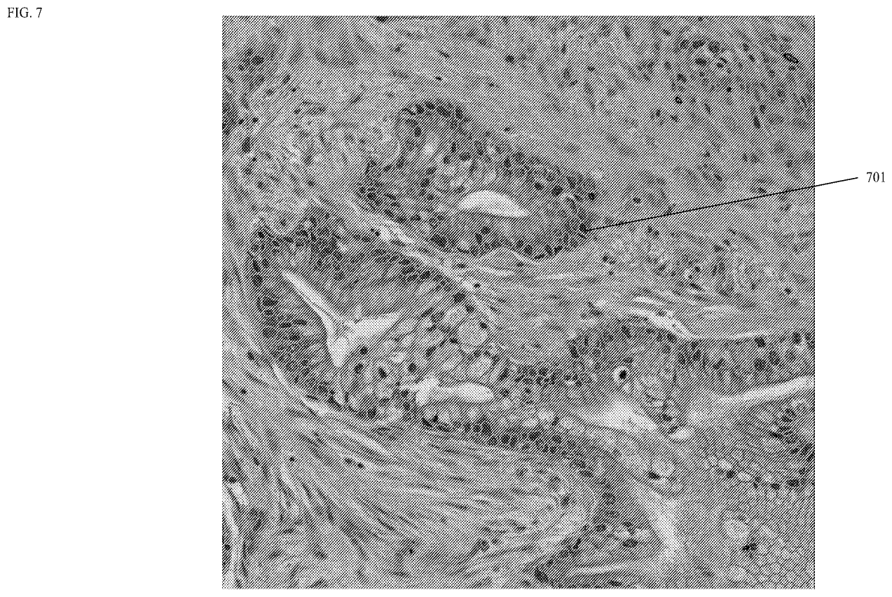

FIG. 7 illustrates nuclei segmentation of a histological image. As illustrated, the platform may detect different types of cells and detect their nuclei. In some embodiments, the platform may provide a user with an augmented histological image in which detected cells and their nuclei are labeled in accordance with their classification (i.e., No-label, Neoplastic, Inflammatory, Connective, Necrosis, Non-neoplastic). FIG. 7 illustrates detected cells 701 and their nuclei.

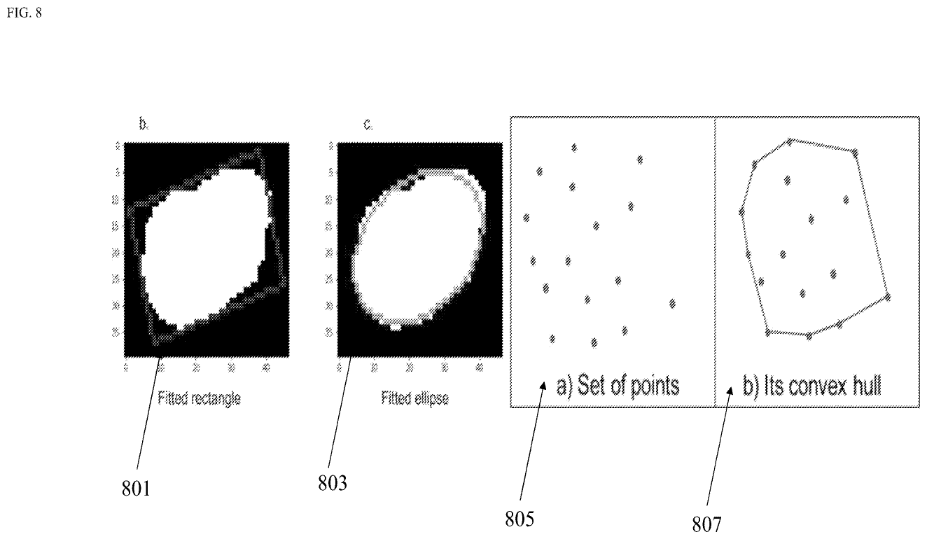

FIG. 8 illustrates how the artificial intelligence based platform may identify and classify morphological features and nuclei automatically into five classes. For example, identified nuclei may be fitted and compared across a rectangle 801 or an ellipse 803. When compared to a fitted rectangle 801 the feature may be characterized based on its rotation angle, height, and width. When compared to a fitted ellipse 803, morphological features may be characterized based on their short axis, long axis, and perimeter. Additionally, a set of points can be characterized by the shape of their grouping 805 and particularly, convexity or concavity 807, and the corresponding hull area and hull perimeter.

### Results

The V-FFX and V-GNP signatures were found to be significantly associated with treatment outcomes stratified in the respective test sets (log-rank test, V-FFX: p=0.046, V-GNP: p=0.004). 29 of 55 patients tested positive for only one of either V-FFX and V-GNP signatures. Kaplan-Meier analysis demonstrated robust separation with hazard ratios for the V-FFX and V-GNP signatures of 3.01 (95% CI: 0.96, 9.45) and 4.81 (95% CI: 1.74, 13.3). DSS at 12 months for patients in V-FFX positive vs negative groups were 88% (8/9) vs 50% (7/14). DSS at 12 months for patients in V-GNP positive vs negative groups were 66% (8/12) vs 15% (2/13).

### Platform Evaluation

Further, as illustrated in Table 1 the prognostic value of risk assessment model applied by the platform described herein was assessed by comparing the disease specific survival percentage of the risk categories output by the model when a multivariate Cox proportional hazards model was used to stratify outcomes under conventional first line chemotherapies.

TABLE 1

| Cohort | DSS % at 12 months | DSS Fraction at 12 months |
|---|---|---|
| V-FFX positive | 88% | 8/9 |
| V-FFX negative | 50% | 7/14 |
| V-GNP positive | 66% | 8/12 |
| V-GNP negative | 15% | 2/13 |

Accordingly, an artificial intelligence based pathology platform such as platform 100 of FIG. 1 may be used for predicting patient outcomes for a given treatment regimen related to pancreatic cancer. In particular, the artificial intelligence based pathology platform generated two signatures (i.e., V-FFX and V-GNP morphological signatures) each of which were strongly associated with successful treatment outcomes for first-line FFX and GNP. Accordingly, the artificial intelligence based pathology platform can aid in the selection of first-line treatment for mPDAC patients.

### Example #2: Development of an Artificial Intelligence-Derived Histologic Signature Associated with Adjuvant Gemcitabine Treatment Outcomes in Pancreatic Cancer

### Overview

Conventionally, pancreatic ductal adenocarcinoma (PDAC) does not have predictive markers that are indicative of response to therapies. Accordingly, there was a need for the development of technologies that can accurately access samples from PDAC patients and determine best courses of treatment. Because PDAC does not have predictive markers, histology is essential to the diagnosis of and treatment of PDAC, as the cellular morphology and stromal characteristics observed in a histological sample may provide information regarding tumor biology and the tumor microenvironment that may aid in selecting a treatment.

In one example, an artificial intelligence (AI) approach to histologic feature examination extracted a signature predictive of disease-specific survival (DSS) in PDAC patients receiving adjuvant gemcitabine after surgical resection. Utilizing an AI based platform analogous to the platform 100 of FIG. 1, a histologic signature strongly associated with outcomes following adjuvant gemcitabine was determined. The AI platform then applied the determined histologic signature to samples taken from pancreatic cancer patients in order to provide recommendations on therapies based on expected outcomes. The disclosed methods provide advantages in determining treatment plans when compared to previously developed transcriptomic classification systems.

Once a histological signature was determined, experimental data externally validated this signature in an independent cohort of patients treated with adjuvant gemcitabine (n=46). Additionally, experimental data indicated that the signature does not stratify survival outcomes in a third cohort of untreated patients (n=161), suggesting that the signature is specifically predictive of treatment-related outcomes but not generally prognostic. Accordingly, the AI based platform described herein may assist in the development of actionable markers in other clinical settings where few biomarkers currently exist.

### Clinical Problem

The prognosis for patients diagnosed with localized pancreatic ductal adenocarcinoma (PDAC) remains poor even after successful surgical resection. Adjuvant chemotherapy regimens, including modified FOLFIRINOX (5-fluorouracil, irinotecan, and oxaliplatin) and gemcitabine-based regimens, have improved overall survival (OS) when compared to observation, but most patients still experience disease recurrence within two years.

Increasingly, neoadjuvant chemotherapy with or without additional post-operative chemotherapy is being utilized, though the optimal regimen or sequence of regimens remains uncertain. Intense study of PDAC tumor genomics has revealed several distinct and reproducible transcriptomic profiles, but to date there are no validated predictive biomarkers to guide recommendation of one chemotherapy regimen over another in clinical practice. Accordingly, there remains a need for improved predictive PDAC tumor biomarkers that can prospectively identify patients most likely to benefit from existing chemotherapy regimens using bioanalytes available in the standard of care setting.

The disclosed AI based pathology platform provides advanced scanning and computational analysis of digitized whole slide images which has created an opportunity for the discovery and exploitation of novel, sub-visual morphologic biomarkers. The AI based pathology platform can identify quantified morphologic features with novel associations to patient outcomes. Quantitative morphometric analyses is used to uncover histologic features associated with response to a particular treatment when a dataset includes patients treated with a specific agent, and outcomes are known. The disclosed AI based pathology platform may include deep learning algorithms that are configured to rapidly segment and classify individual cell types. In some embodiments the AI based pathology platform uses deep learning in conjunction with morphometric analysis to identify novel associations between specific cellular compartments in the tumor microenvironment and responses to treatment, which enables identification of treatment-specific biomarkers, such as an association between the spatial arrangement of tumor infiltrating lymphocytes and immune checkpoint inhibitor response.

As discussed with respect to this experiment, the AI based pathology platform was used to quantitatively extract morphologic features using deep learning in order to identify a histologic signature associated with outcomes following administration of a particular adjuvant treatment (i.e., gemcitabine) in resected PDAC. Additional experimentation explored the degree to which an AI-derived histologic signature was associated with adjuvant gemcitabine treatment outcomes and compared its performance to existing transcriptomic subtypes. Further experimentation examined the performance of the histologic signature determined by the AI based pathology platform in an external cohort of patients who underwent resection of PDAC followed by adjuvant gemcitabine to determine whether results could be generalized. Additionally, experimental results were validated by comparing the AI determined histologic signature in another cohort where patients received no adjuvant treatment to ensure that the association with disease-related outcomes was predictive (specific to treatment) and not prognostic (related to the underlying disease process).

### Methods

Data from three cohorts of patients were utilized for this experiment: a cohort of 93 patients forming a training data set, a cohort of 46 patients forming a first external data set, and a cohort of 161 patients forming a second external data set. The training data set was identified by selecting PDAC patients who had received adjuvant gemcitabine and no 5-fluorouracil. The training data set included available histopathologic images, data on disease specific survival, and demographic details. From the available histopathologic images, the image identified as the diagnostic slide was used for image analysis by the AI-based pathology platform.

RNASeq classifications for the first external data set were obtained. As will be discussed below, the dataset was randomly split in half into a training and test set with no overlap between groups. To be able to make direct comparisons to existing RNA subtypes, 8 patients without RNASeq data were removed from the test set for a sub-group analysis (discussed below). Tests of associations between the histologic signature and RNASeq clusters were made using the 79 patients from the entire training set who had RNASeq data available.

The first external data set represented a set of consecutive 24 patients treated with resection and adjuvant gemcitabine. Clinical data was obtained via manual chart review of the electronic medical record. Digitally scanned tissue microarray specimens were used for image analysis. The cores were 1 mm and were obtained from formalin-fixed, paraffin-embedded (FFPE) samples of extra portions of surgical resections. Whole tissue resection specimens were not available for analysis for this study.

The second external data set included 161 patients who underwent pancreaticoduodenectomy between 1978 and 2008 as part of a previously described study. Whole tissue sections stained with hematoxylin and eosin from these patients were scanned using the ImageScope 12.2 (manufactured by Leica Biosystems, Wetzlar, Germany).

Scanned histologic images were analyzed through an AI-based pathology platform in accordance with the systems and methods described herein. The process included tissue segmentation, nuclei segmentation, and finally geometric feature extraction. Tissue was segmented via color-based thresholding to remove empty regions of the slide. Patches of size 2132×2132 were extracted from tissue regions and a validated deep learning model developed was used to segment and classify each nucleus automatically into five classes (i.e., Neoplastic, Connective, No-Neoplastic/Epithelial, Necrotic, Inflammatory). Descriptive morphometric features were then computed for each nucleus. Geometric features were then aggregated first at the patch, and subsequently at the patient level using summary statistics including the mean, standard deviation, skewness and kurtosis to produce the final feature vector for a patient. This feature vector was used as the input to a cox proportional hazards model that used the least absolute shrinkage and selection operator to identify the most correlated features with DSS along with their coefficients on the training set.

Phase One: Utilizing an AI Based Pathology Platform to Develop a Histologic signature known as the visual pancreatic gemcitabine signature (VPG) from a training set From The Cancer Genome Atlas (TCGA).

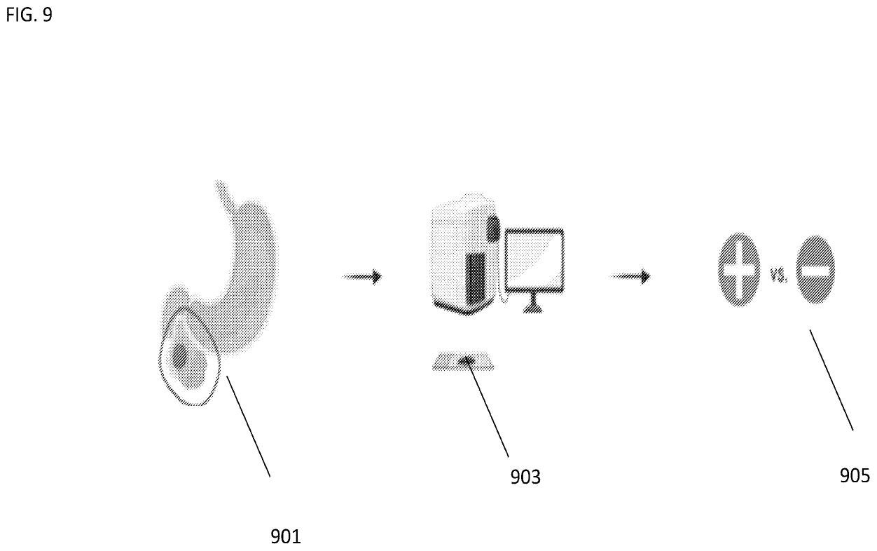

FIG. 9 provides an illustration of a process to construct a histologic signature associated with disease-specific survival (DSS) after adjuvant gemcitabine. A dataset of scanned whole slide images and the associated clinical data from a cohort of 93 PDAC patients treated with adjuvant gemcitabine in The Cancer Genome Atlas (TCGA) was analyzed. The process to construct a histologic signature includes obtaining a slide 901, digitizing a slide 903, and determining the presence or absence of a histologic biomarker 905. Slides may be obtained in step 901 by resection of pancreatic ductal adenocarcinoma (PDAC). The slides may then then be digitized in step 903 by scanning hematoxylin and eosin-stained resection specimens for whole slide images and subsequently performing digital pathology analysis. In step 905, an AI based pathology platform such as the platform described herein is used to identify the presence or absence of a histologic biomarker associated with improved outcomes following adjuvant gemcitabine therapies.

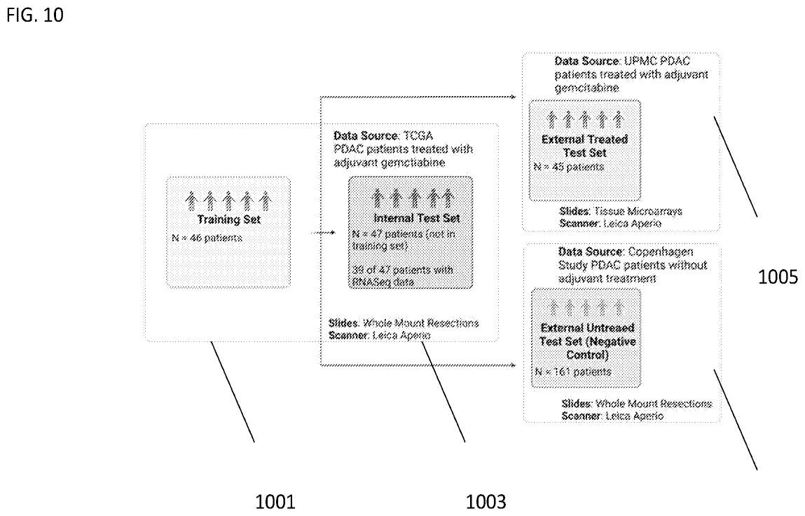

FIG. 10 illustrates sources of training and validation data for the signature created by the AI based pathology platform. As illustrated, the AI based pathology platform was trained using a training set 1001 of 46 patients from the TCGA data set. A remaining subset of patients, a validation subset 1003 of 47 patients, who were not included in the training set but were treated with adjuvant gemcitabine were used to validate the signatures created by the AI based pathology platform. In the illustrated experiment, patients were randomly assigned to the training or test sets, and characteristics were similar between the two groups as summarized in Table 2. Table 2 provides the clinical characteristics of the training and test sets from the TCGA. As illustrated, patients were randomly divided between the training and testing sets. Further, the P-values in Table 2 correspond to chi-squared tests run with the exception of the variable age, for which a Wilcoxon Rank Sum Test was run.

TABLE 2

| | | Training | | Test | | |
|---|---|---|---|---|---|---|
| n | | 46 | | 47 | | |
| Age, Median (IQR) | | 65 | (56, 74.8) | 65 | (60, 71) | p = 0.65 |
| Sex (%) | Female | 23 | (50) | 17 | (36) | p = 0.26 |
| | Male | 23 | (50) | 30 | (64) | |
| Tumor Grade (%) | G1 | 2 | (4) | 10 | (21) | p = 0.09 |
| | G2 | 30 | (65) | 22 | (47) | |
| | G3 | 13 | (28) | 14 | (30) | |
| | G4 | 1 | (2) | 1 | (2) | |
| Adjuvant Regimen | Gemcitabine alone | 43 | (93) | 45 | (96) | p = 0.98 |
| Received (%) | Gemcitabine in combination | 3 | (7) | 2 | (4) | |
| | another agent | | | | | |
| Length of Adjuvant | <3 months | 19 | (41) | 27 | (57) | p = 0.26 |
| Therapy (%) | 3-6 months | 10 | (22) | 9 | (19) | |
| | >6 months | 17 | (37) | 11 | (23) | |

Subsequently, the performance of the signature in stratifying patients was assessed in two external validation cohorts. A first external cohort 1005 included 45 patients who underwent PDAC resection followed by gemcitabine treatment for whom digitally scanned tissue microarrays of tumor specimens were available. A second external cohort 1007 included 161 patients, whose tumors were resected between 1978 and 2008, when adjuvant treatment was not administered as part of the standard of care.

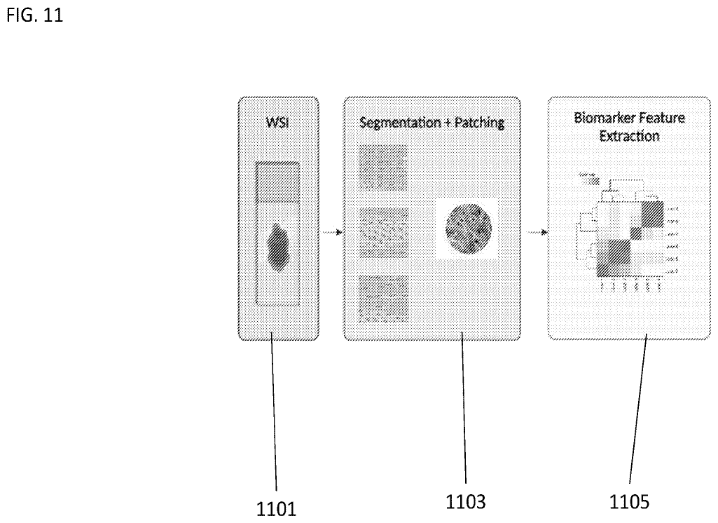

FIG. 11 shows a process for image analysis AI based pathology platform. As illustrated in FIG. 11, a histologic signature capable of stratifying patients by disease-related outcomes following adjuvant gemcitabine was generated by the AI based pathology platform by applying the AI based pathology platform to data from the TCGA cohort. In particular whole slide images 1101 were segmented and patched 1103 in order to extract biomarker features 1105 and create a histologic signature. As illustrated segmentation and patching 1103 may involve nuclei segmentation, extraction of a plurality of features describing nuclear morphology (e.g., 816 features), feature selection using least absolute shrinkage and selection operator (LASSO) regression, training a cox proportional hazards model incorporating selected features in a training set of 46 patients corresponding to training set 1001 of FIG. 10, and testing the performance of the signature in the test set of remaining 47 patients corresponding to validation subset 1003 of FIG. 10.

The process illustrated in FIG. 11 results in a histologic signature referred to as a visual pancreatic gemcitabine (VPG) signature (VPG). The VPG signature incorporates a single feature that describes the variance in nuclear morphology among neoplastic cells of a tumor. VPG positivity may be defined using a threshold determined by the median patient in the training set, with positive patients defined by feature quantification greater than the median patient, and negative patients defined by feature quantification lower than the median patient.

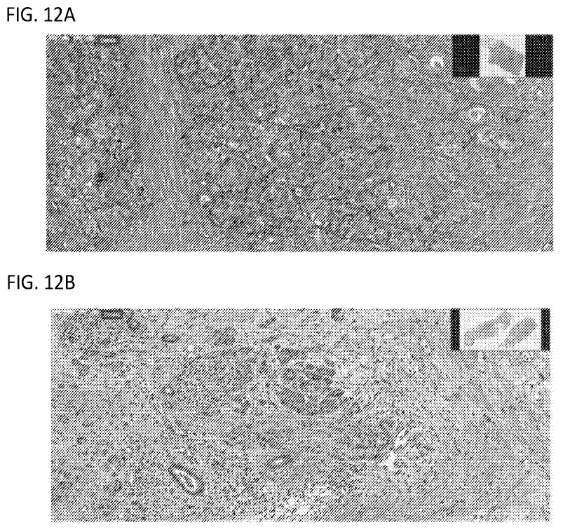

FIG. 12A provides an illustration of samples determined by the AI based pathology platform to have a positive visual pancreatic gemcitabine (VPG) signature 1201. FIG. 12B provides an illustration of samples determined by the AI based pathology platform to have a negative VPG signature 1203. As illustrated in FIGS. 12A and 12B, the feature contributing to the VPG signature describes variation in nuclear morphology and demonstrates significant variation visually. Both of the slides illustrated in FIGS. 12A and 12B correspond to patients with tumor grade of G3, where cancer cells and tissue look very abnormal without an architectural structure or pattern.

Phase Two: Experimental Data Demonstrates that the VPG Signature Stratifies Dss Outcomes Following Adjuvant Gemcitabine Treatment.

The histological signature determined by the AI based pathology platform was tested on a validation subset 1003 (of FIG. 10) of 47 patients from the TCGA cohort. In this validation set 1003, the characteristics of the patients found to have a positive VPG signature (n=23) did not differ from those with a negative VPG signature (n=23). These characteristics include age, sex, or grade of tumor, and duration of adjuvant gemcitabine therapy. Clinical characteristics of the validation subset 1003 are summarized in Table 3. As shown in Table 3, clinical characteristics among patients in the TCGA test set who had a positive VPG signature were compared to those with a negative VPG signature. Table 3 also displays the P-values that correspond to chi-squared tests run with the exception of the variable age, for which a Wilcoxon Rank Sum Test was run.

TABLE 3

| | | Signature+ | | Signature− | | |
|---|---|---|---|---|---|---|
| n | | 23 | | 24 | | |
| Age, Median (IQR) | | 67 | (59, 71) | 65 | (62, 71) | p = 0.72 |
| Sex (%) | Female | 7 | (30) | 10 | (42) | p = 0.62 |
| | Male | 14 | (70) | 16 | (58) | |
| Tumor Grade (%) | G1 | 4 | (17) | 6 | (25) | p = 0.60 |
| | G2 | 12 | (52) | 10 | (42) | |
| | G3 | 6 | (26) | 8 | (33) | |
| | G4 | 1 | (4) | 0 | (0) | |
| Adjuvant Regimen | Gemcitabine alone | 22 | (96) | 23 | (96) | p = 1 |
| Received (%) | Gemcitabine in combination | 1 | (4) | 1 | (4) | |
| | another agent | | | | | |
| Length of Adjuvant | <3 months | 11 | (48) | 16 | (67) | p = 0.40 |
| Therapy (%) | 3-6 months | 7 | (30) | 4 | (17) | |
| | >6 months | 5 | (21) | 4 | (17) | |

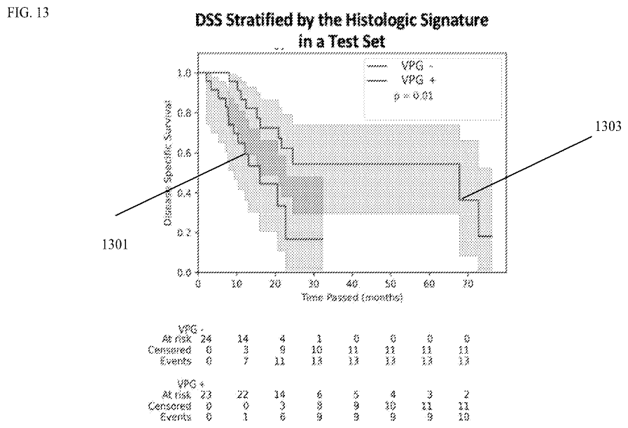

FIG. 13 illustrates experimental data obtained by applying the AI based pathology platform on the validation subset 1003 of FIG. 10. In particular, FIG. 13 indicates that the VPG signature is strongly associated with Disease Specific Survival (DSS) in the internal validation cohort (log-rank P≤0.001). Further, the hazard ratio for death for negative VPG signature patients was 2.94 (95% CI: 1.21, 7.14). Positive VPG signature patients 1303 had a median DSS of 67.9 months (95% CI: [16.2, not reached]) while negative VPG signature patients 1301 had a median DSS of 16.0 months (95% CI: [9.3, 22.8]).

### Phase Three: Validation of Use of VPG Signature to Stratify Outcomes Following Adjuvant Gemcitabine in Comparison to Conventional RNAseq Classification Systems.

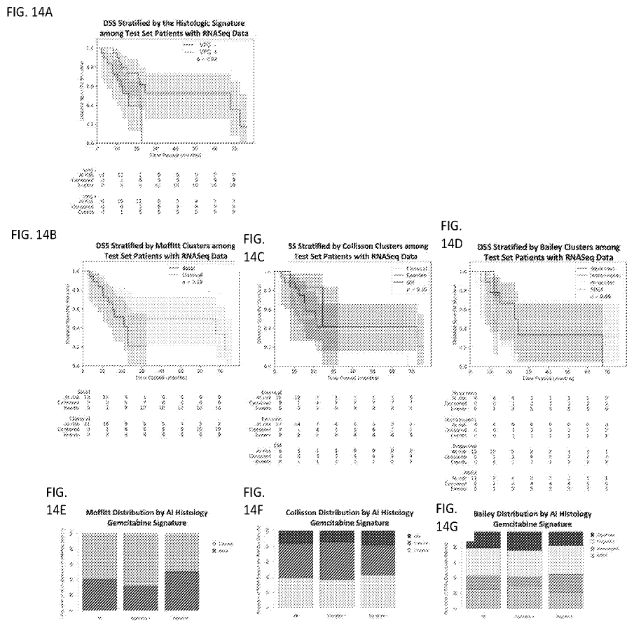

Conventional methods for determining treatment therapies for pancreatic cancer patients include the use of RNAseq classification systems. VPG signatures generated by the AI based pathology platform described herein and used for determination of treatment therapies were compared to RNAseq classification systems experimentally. As illustrated in FIG. 14A, a sub-group of 39 patients within the validation subset also had RNAseq data and classification available. Accordingly, performance of the VPG signatures could be compared to RNAseq data and classification. Also illustrated in FIG. 14A are Kaplan-Meier estimators among positive VPG signature and negative VPG signature patients which indicate that DSS differed between the two groups (log-rank test p-value=0.02, positive VPG signature median DSS=67.9 months, 95% CI [15.3, not reached] when compared with negative VPG signature median DSS=16.0 months [8.0, 22.8]).

FIGS. 14B-14D illustrate that the traditional RNAseq classification approaches are unable to account for differences in DSS between stratifications. For example, the Log-rank test p-values for Moffit classification approach was p=0.28 as illustrated in FIG. 14B. The Log-rank test p-values for Collisson classification approach was p=0.3 as illustrated in FIG. 14C. The Log-rank test p-values for Bailey classification approach was p=0.96 as illustrated in FIG. 14B. Additionally, FIGS. 14E-G illustrate results from experiments assessing whether there was an association between the signature and the RNAseq clusters by examining the classifications of the signature and RNAseq clusters among all patients in the TCGA cohort with RNAseq data (n=79 patients). In this group, the chi squared values describing the association between the presence of the signature and the Moffitt (FIG. 14E), Collisson (FIG. 14F), and Bailey (FIG. 14G) RNAseq clusters were 2.03 (p=0.15), 0.71 (p=0.70), and 2.36 (p=0.50) respectively, suggesting that the signature is not associated with the RNAseq clusters.

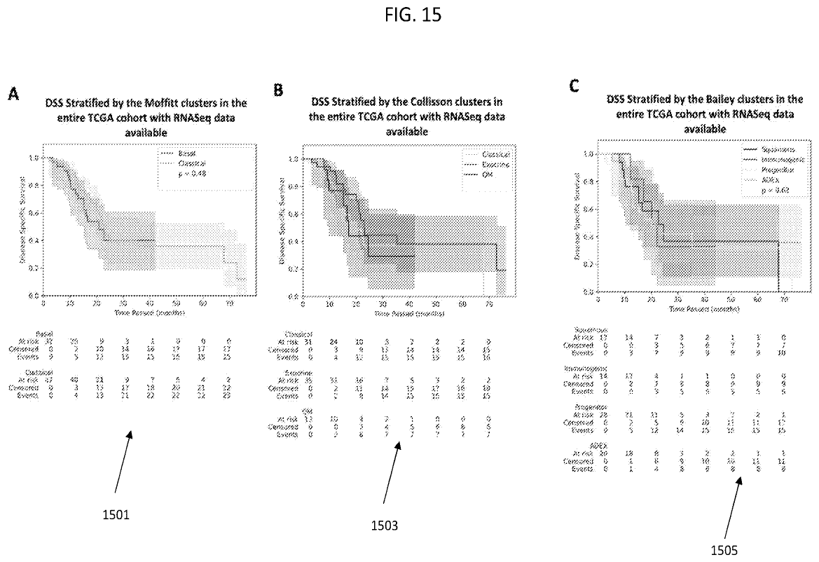

To confirm that the stratification of the RNASeq clusters was not unique to the patients in the test set, the same clusters were stratified across the entire gemcitabine-treated TCGA dataset of patients with RNAseq data available (n=79). FIG. 15 illustrates results from this stratification and further illustrates that it is not possible to observe a difference in survival outcomes across clusters. In particular, FIG. 15 shows that RNASeq clusters do not stratify patients by DSS following adjuvant gemcitabine across the entire gemcitabine-treated TCGA dataset (n=92). Three Kaplan Meier curves describing DSS among all patients in the TCGA cohort with RNASeq data available (n=79) are shown when stratified by Moffitt clusters 1501, Collisson clusters 1503, and Bailey clusters 1505.

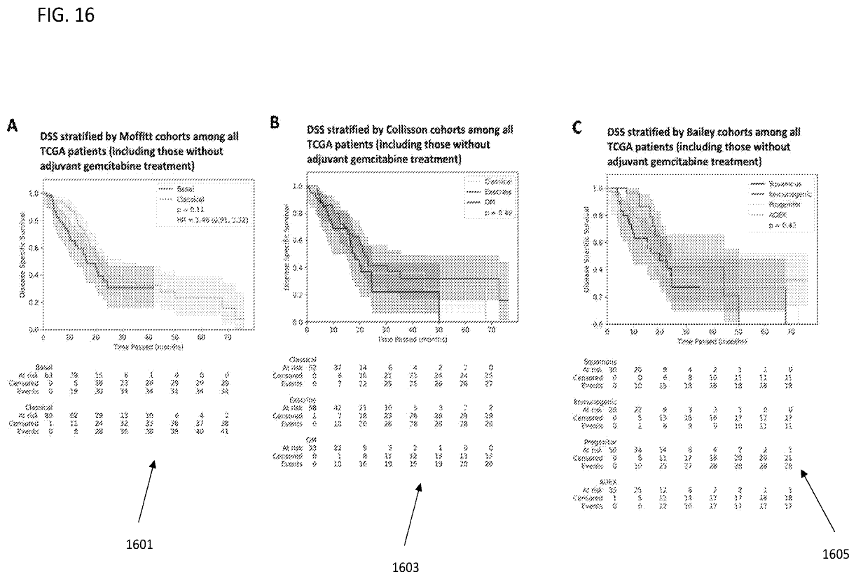

Additionally, as illustrated in FIG. 16, the RNAseq cohorts also did not correlate with DSS among all TCGA patients with RNAseq data, including those who did not receive adjuvant treatment or received an adjuvant therapy other than FOLFIRINOX, though there was a trend toward significance among Moffitt subsets. RNASeq clusters do not stratify patients by DSS across the entire TCGA dataset regardless of adjuvant treatment (n=143). Three Kaplan Meier curves describing DSS among all patients in the TCGA cohort with RNASeq data available regardless of adjuvant treatment received (n=143) when stratified by Moffitt clusters 1601, Collisson clusters 1603, and Bailey clusters 1605.

### Phase Four: Validation of the VPG Signature in an External Cohort of Gemcitabine-Treated Patients.

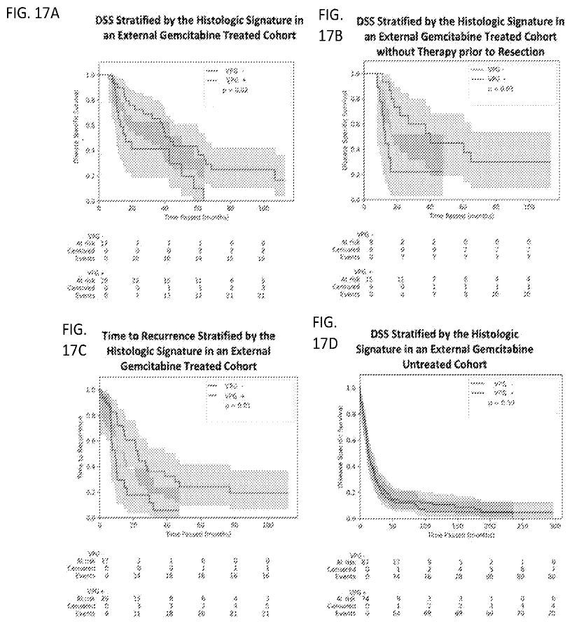

To investigate performance beyond the TCGA dataset, experiments applying the model in a retrospective cohort of adjuvant-gemcitabine-treated patients and a retrospective cohort of untreated patients were performed. As illustrated in FIGS. 17A-17D, the VPG signature generalizes to external cohorts of gemcitabine-treated patients but not untreated patients.

FIG. 17A illustrates experimental data including Kaplan Meier curves describing DSS among patients receiving adjuvant gemcitabine-based therapy in the cohort (n=46) when stratified by the histologic signature. The p-value (p=0.02) corresponds to the log-rank test. Median DSS of positive VPG signature patients was 43.1 months (95% CI: 26.8, 63.9) as compared to the median DSS of negative VPG signature patients which was 16 months (95% CI: 10.8, 50.1). The DSS in patients identified as having a positive VPG signature was superior to the DSS of those who were identified as having a negative VPG signature. As illustrated in Table 4, the clinical characteristics of patients with and without the VPG signature was similar. The P-values in Table 4 correspond to the P-values correspond to chi-squared tests run with the exception of the variable age, for which a Wilcoxon Rank Sum Test was run.

TABLE 4

| | | Signature+ | | Signature− | | |
|---|---|---|---|---|---|---|
| n | | 29 | | 17 | | |
| Age, Median (IQR) | | 60 | (55, 71) | 66 | (54, 71) | p = 0.59 |
| Sex (%) | Female | 13 | (45) | 5 | (29) | p = 0.47 |
| | Male | 16 | (55) | 12 | (71) | |
| ECOG (%) | 0 | 6 | (21) | 1 | (6) | p = 0.37 |
| | 1 | 4 | (14) | 2 | (12) | |
| | Not available | 19 | (66) | 14 | (82) | |
| Tumor Grade (%) | G1 | 3 | (10) | 0 | (0) | p = 0.05 |
| | G2 | 23 | (79) | 10 | (59) | |
| | G2-3 | 0 | (0) | 2 | (12) | |
| | G3 | 3 | (10) | 5 | (29) | |
| Neoadjuvant Therapy | None | 15 | (52) | 9 | (53) | p = 0.67 |
| Received | 5-FU Backbone | 6 | (21) | 5 | (29) | |
| | Gemcitabine | 8 | (28) | 3 | (18) | |
| | Backbone | | | | | |
| Adjuvant Regimen | Gemcitabine alone | 21 | (72) | 11 | (65) | p = 0.45 |
| Received (%) | Gemcitabine in combination | 6 | (21) | 6 | (35) | |
| | with another agent | | | | | |
| | Gemcitabine in combination | 2 | (7) | 0 | (0) | |
| | with radiation | | | | | |
| Length of Adjuvant | <3 months | 6 | (21) | 4 | (24) | p = 0.96 |
| Therapy (%) | 3-6 months | 19 | (66) | 10 | (59) | |
| | >6 months | 3 | (10) | 2 | (12) | |
| | Date not available | 1 | (3) | 1 | (6) | |

FIG. 17B illustrates experimental data including Kaplan Meier curves describing DSS among patients, who had received no therapy prior to surgery (n=24) (log-rank test p=0.03). Median DSS of positive VPG signature patients was 40.2 months (95% CI: 16.4, not reached) when compared to median DSS of negative VPG signature patients was 12.9 months (95% CI: 8.1, not reached). As illustrated, 22 of 46 patients in the cohort had received neoadjuvant chemotherapy prior to resection, and in a sub-group analysis of the 24 patients without neoadjuvant chemotherapy, the DSS remained significantly different between positive VPG signature patients and negative VPG signature patients.

FIG. 17C illustrates experimental data including Kaplan Meier curves describing time to recurrence among patients who received adjuvant gemcitabine-based therapy (n=46) (log-rank test p=0.01). Median time to recurrence of positive VPG signature patients was 22.6 months (95% CI: 14.1, 44.8) in comparison to the median time to recurrence among negative VPG signature patients of 9.1 months (95% CI: 6.4, 14.7). FIG. 17C illustrates that when using the clinically meaningful alternate endpoint of time to recurrence, there was still a difference in the outcomes between positive VPG signature and negative VPG signature patients.

FIG. 17D illustrates experimental data including Kaplan Meier curves describing DSS among patients in the cohort who were untreated (log-rank test p=0.59; positive VPG signature median DSS: 13.2 months [10.4, 19.8], negative VPG signature median DSS: 12.3 [10.4, 19.8]). FIG. 17D demonstrates the independent effect of the VPG signature in the cohort, a multivariate Cox proportional hazards model of DSS including the VPG signature and the covariates of age, performance status as defined by ECOG score, and CA19-9 level before treatment was applied. In the model, the VPG signature was statistically significantly associated with improved DSS (HR=0.41 [0.19, 0.88], p=0.02), along with the clinical covariate of age. In contrast, in the experimental data for the cohort of untreated patients, the log-rank test comparing DSS between positive VPG signature and negative VPG signature patients showed no association (Log-rank test p=0.59), suggesting that the VPG signature was not a prognostic factor for untreated tumors. A summary of the clinical characteristics of the experimental data set is presented in Table 5. As shown in Table 5, the patients in the clinical dataset were of similar age, sex, and tumor grade. The P-values in Table 5 correspond to chi-squared tests run with the exception of the variable age, for which a Wilcoxon Rank Sum Test was run.

TABLE 5

| | | Signature+ | | Signature− | | |
|---|---|---|---|---|---|---|
| n | | 74 | | 87 | | |
| Age, Median (IQR) | | 62 | (53, 69) | 63 | (57, 69) | p = 0.54 |
| Sex (%) | Female | 36 | (49) | 40 | (46) | p = 0.86 |
| | Male | 38 | (51) | 47 | (54) | |
| Tumor Grade (%) | G0 | 0 | (0) | 1 | (1) | p = 0.09 |
| | G1 | 27 | (36) | 19 | (22) | |
| | G2 | 15 | (20) | 24 | (28) | |
| | G3 | 32 | (43) | 39 | (45) | |
| | G4 | 0 | (0) | 4 | (5) | |

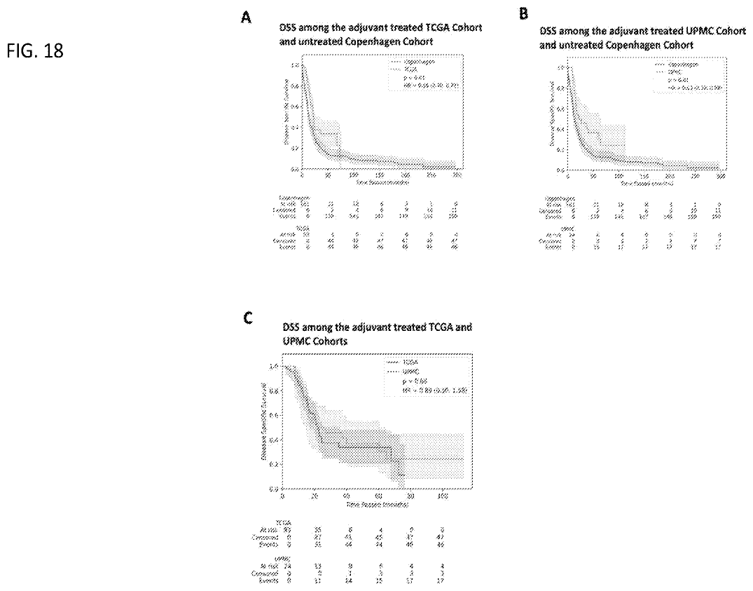

FIGS. 18A-18C illustrate experimental data that indicates that the DSS in the untreated experimental cohort did differ from the adjuvant gemcitabine-treated cohorts. FIG. 18A provides a Kaplan Meier curve describing DSS among all adjuvant gemcitabine-treated cohort patients (n=93) and all untreated cohort patients (n=161). The p-value for the log-rank test is <0.01. FIG. 18B provides a Kaplan Meier curve describing DSS among all adjuvant gemcitabine-treated cohort patients (n=24) and all untreated cohort patients (n=161). The p-value for the log-rank test is 0.01. FIG. 18C Kaplan Meier curves describing DSS among all adjuvant gemcitabine-treated cohort patients (n=161) and all untreated cohort patients (n=24). The p-value for the log-rank test is 0.68.

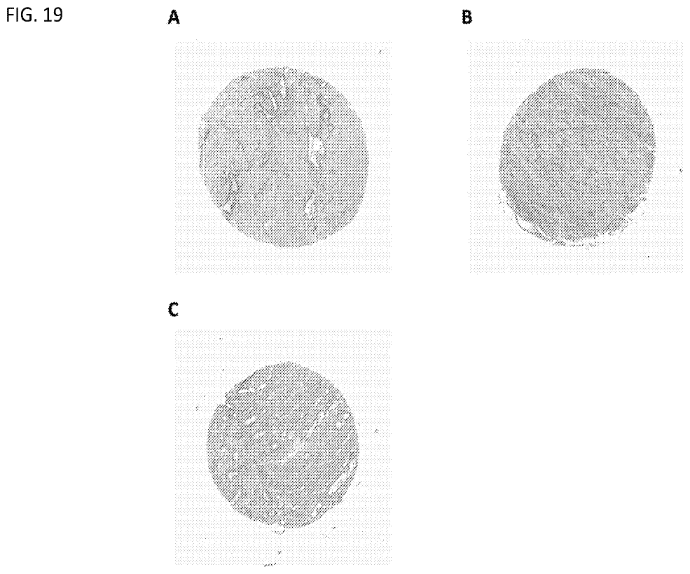

FIG. 19 provides three representative examples of scanned images of tissue microarray samples of the external validation set.

### Results

An AI digital pathology platform identified a histology-based morphological signature associated with treatment outcomes following post-operative treatment with gemcitabine in patients with resected PDAC. Experimental data indicated that the VPG signature developed by the AI digital pathology platform successfully stratified patient outcomes (i.e., Disease specific survival or DSS) following the administration of adjuvant gemcitabine. The systems and methods described herein were validated in an external cohort of patients with the additional endpoint of time to recurrence.

The VPG signature generated by the AI based digital pathology platform provides immense clinical value. First, given the significant difference in disease-related outcomes between positive VPG signature patients and negative VPG signature patients across multiple cohorts tested in this study, this signature may help clinicians identify which patients will benefit from gemcitabine-based therapy after resection. Further, the differences in outcomes between positive VPG signature patients and negative VPG signature patients across multiple cohorts tested in this study point toward its being a predictive biomarker in a population where currently none exists.

Second, the VPG signature generated by the AI based digital pathology platform discussed herein may, on a larger scale, improve the process of designing clinical trials for resected PDAC. Randomizing a large cohort of patients with molecularly heterogeneous tumors to treatment arms without predefined biomarkers compromises results and leads to inefficiency as well as a waste of precious resources.

Third, the VPG signature generated by the AI based digital pathology platform discussed herein can be tested and refined for application in other clinical settings, for example in metastatic or borderline resectable PDAC, when FOLFIRINOX and gemcitabine/nab-paclitaxel are both acceptable frontline regimens without a reliable predictive biomarker to help clinicians to recommend one over the other.

Since the AI based digital pathology platform is able to generate the VPG signature from images of H&E slides, which are routinely generated for all patients with PDAC, no additional tissue or complex molecular testing is required. Subsequently, both turnaround time and cost are much lower for the methods described herein than one would expect for predictive biomarker testing. Further, the experimental validation studies, in which tissue microarray specimens were used to generate images shows that feature extraction is feasible across different techniques of tissue preparation.

The favorable performance of the signature generated by the AI-based pathology platform, when compared to existing RNAseq-based clusters, in stratifying disease-related outcomes in patients treated with gemcitabine validates pursuing modalities other than genomics and transcriptomics as potential predictive biomarkers. PDAC RNAseq subtypes were developed to provide an improved molecular taxonomy of PDAC and, in turn, inform therapeutic development. These subtypes have been correlated with prognosis, including a recent study of the Basal and Classical subtypes in a multicenter trial, however their association with prognosis was never previously assessed.

Prior studies have indicated that that the Moffitt Basal and Classical subtypes have different outcomes following first-line chemotherapy. Subsequent analysis from the same studies revealed that the Basal and Classical subtypes stratify patients who received modified FOLFIRINOX, but not those who received gemcitabine plus nab-paclitaxel. While the prior studies featured patients with metastatic disease, experimental data presented herein showed similar results for gemcitabine-treated patients after surgical resection and confirmed that the Bailey and Collisson systems fail to stratify outcomes among gemcitabine-treated patients. The experimental results presented herein suggest that prevailing molecular taxonomies do not provide adequate predictive stratification for patients treated with gemcitabine-based regimens. Further, previously designated subtypes did not appear to be prognostic, even when analyzing other treatments. Possible explanations include a smaller proportion of patients in the data set who received fluoropyrimidine-based therapy or differences between treatment effect in the adjuvant and metastatic settings. Regardless, the performance of the VPG signature generated by the AI-based pathology platform validates the capacity for digital pathology approaches to identify biomarkers predictive of treatment response when existing molecular approaches have not been proven to do so. Further, the ability to construct such a signature in a limited size data set (e.g., in a training set of fewer than fifty patients) illustrates that clinically meaningful tools can be generated from relatively small cohorts of patients and that the same technology can be applied to other clinical contexts.

Additionally, the experimental data suggests that the AI-generated VPG signature is not a prognostic marker of a tumor's underlying biology. Instead, the validation of the VPG signature in an external cohort of gemcitabine-treated patients in combination with the data from the untreated cohort, suggest that the VPG signature is likely specific to chemotherapy treatment.

In summary, the experimental data identifies a histologic signature generated by an AI based pathology platform that stratifies disease-related outcomes among patients who have received adjuvant gemcitabine after resection of PDAC, where transcriptional profiling-based subtyping fails to do so. This signature may provide a clinically applicable predictive biomarker for PDAC.

Although applications for pancreatic cancer are described herein, it is envisioned that the AT-based pathology platform and the imaging analysis platform underlying this signature may be generalized to other clinical settings, thereby facilitating the emergence of biomarkers to predict treatment response in diseases for which few actionable biomarkers currently exist.

### Example #3: Use of an AT Based Pathology Platform in Predicting Gemcitabine Response in the Adjuvant Treatment of Resected Pancreatic Ductal Adenocarcinoma

### Clinical Problem

Adjuvant chemotherapy improves survival following resection of pancreatic ductal adenocarcinoma (PDAC). A modified fluorouracil/irinotecan/oxaliplatin regimen (mFOLFIRINOX) has demonstrated improved disease free survival and overall survival, though gemcitabine-based monotherapy and gemcitabine plus capecitabine are alternatives in less fit patients. Though there are several proposed biomarkers to guide treatment decisions (e.g., GATA6, hENT1, and GemPred), no biomarker is currently used to guide treatment selection in clinical practice.

An AI-based pathology platform was used to generate a signature of features from digital images of routine histopathology specimens that could identify patients susceptible to routine chemotherapeutic agents.

### Methods

One hundred and thirty-nine (139) whole-slide digitized histological slides corresponding to one hundred and two (102) resected PDAC tumors from a data set were used as a training set. This dataset corresponded to patients that had received either gemcitabine-backbone or 5 FU-backbone chemotherapy as their first-line adjuvant treatment.

An AI-based pathology platform such as the one described herein was used to extract nuclei images from tissue regions using segmentation models and compute geometric features of these nuclei. The subsequent features were then correlated with Disease Specific Survival (DSS) in order to construct a signature associated with treatment benefit. The resulting signature was compared against two board certified pathologists using the grade of the digital slides images to classify patients into above or below average DSS buckets.

### Results

Among quantitative geometric features, a set of area and ellipse features describing nuclei geometry correlated most with response to gemcitabine (RO.4). A second model, a cox proportional hazards model, was applied to the geometric nuclei features and was found to be predictive of response to gemcitabine and achieved a C-index (95% CI) of 0.69 (0.58, 0.79). The pathologist-based baseline model for above and below average DSS had a median DSS of 443 and 461 days respectively. Using the average expected lifetime as the threshold, the model divides patients receiving gemcitabine into two histological subtypes with median DSS of 586 and 394 days respectively (p<0.05). The model appeared specific to gemcitabine. Among patients receiving 5-FU (n=10) there was no statistical significance in median DSS between the subtypes and a c-index of 0.63 (0.27, 1.0).

Accordingly, in this example, the AI based pathology platform described herein utilized routine histopathology to identify features that correlate with treatment outcomes in PDAC with classification performance (c-index:0.69) superior to the validated AJCC treatment prediction tool (0.59). Further, the disclosed AT based pathology platform is able to provide clinically relevant signatures for a backbone treatment even when trained on datasets with adjuvant therapies.

### Example #4: Validation of a Signature Generated by a AI-Pathology Platform

In this example, the signature generated by a AI-pathology platform was validated. In particular, the signature was validated on an external cohort of postoperatively treated PDAC cases. The AI-pathology platform discussed herein allows for the identification of subvisual morphologic features in digital scans of routine histologic slides that are associated with specific treatment responses.

### Clinical Problem

The prognosis for patients diagnosed with pancreatic ductal adenocarcinoma remains poor, even after successful resection. While multiple regimens have proven to improve outcomes following resection, no biomarkers routinely used in clinical practice can predict which regimen is optimal for an individual patient to facilitate a precision medicine approach.

### Methods

Digitized histological hematoxylin and cosin stained tissue microarray blocks corresponding to post-operatively treated resected PDAC patients from 2011-2015 were used in this experiment. Of the 45 patients, 22 were neoadjuvantly treated with either gemcitabine or 5-FIU backbone cytotoxic chemotherapy. Using the histologic images, the AI-based pathology platform extracted nuclei images from tissue regions using segmentation models and computed geometric features of these nuclei. Patients were stratified by the signature previously associated with gemcitabine response in a dataset into low and high risk groups, and Disease Specific Survival (DSS) and Recurrence Free Survival (RFS) was compared between the stratified groups via Kaplan Meier estimators and log-rank test.

### Results

The morphologic signature generated by the AI-based pathology platform and previously found to be associated with gemcitabine treatment response stratified both DSS and RFS in the external cohort (log-rank test, DSS: p=0.03, RFS: p=0.01). A set of features describing variations in nuclear geometry were most correlated with the prediction, with increased variance being associated with higher risk. Kaplan-Meier analysis demonstrated the generated signature was able to separate the cohort robustly with a statistically significant hazard ratio of 0.45 [95% CI 0.22, 0.93] for DSS and 0.39 [95% CI 0.19, 0.77] for RFS. The median DSS was 16 months (95% CI: 10.9, 50.1) in the high risk group and 43 months (95% CI: 26.8, 63.8) in the low risk group, a difference of 27 months. Similarly, the median RFS was 9.1 months (95% CI: 6.1, 14.7) in the high risk group and 22.6 months (95% (C: 14.1, 44.8) in the low risk group, a difference of 13.5 months.

Thus, the AI based pathology platform generated a morphological signature that was previously found to be associated with gemcitabine treatment response and also effectively stratifies patients into low and high risk groups in an external resected PDAC cohort (hazard ratio: 0.45 for DSS, 0.39 for RFS).

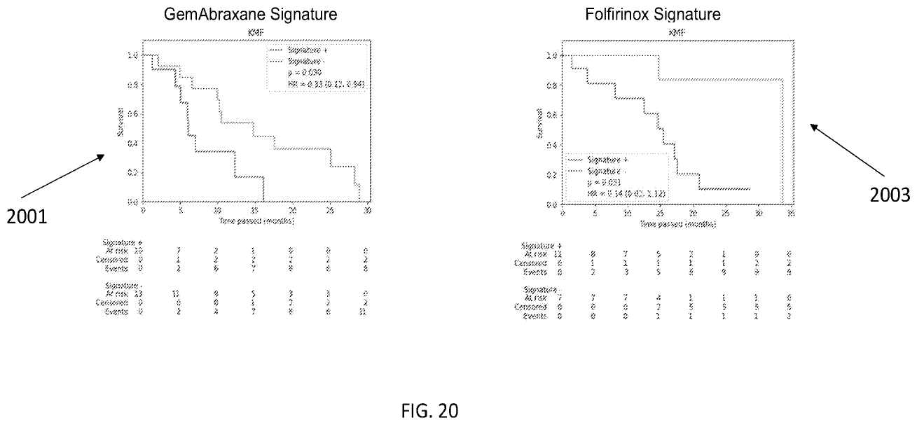

FIG. 20 provides diagrams for experimental results for an artificial intelligence based pathology platform. In particular, the AI-based pathology platform was used to generate a signature corresponding to Gem Abraxane and Folfirinox. As illustrated, a signature generated by the AI-pathology platform related PDAC, generalizes to metastatic PDAC across biopsy sites. In particular, a first set of experimental data 2001 that indicates the DSS in a gemcitabine-abraxane treated is shown in a first Kaplan Meier curve. Additionally, a second set of experimental data 2003 as shown in a second Kaplan Meier curve for a Folfirinox signature, indicates that the AI-pathology platform can be used to generate signatures corresponding to individual treatments. Further, DSS can be stratified based on whether histological data is classified as either positive of negative for the respective treatment signature.

One skilled in the art will appreciate further features and advantages of the invention based on the above-described embodiments. Accordingly, the invention is not be limited by what has been particularly shown and described, except as indicated by the appended claims.

We claim:

1. A method performed by at least one processor for predicting outcomes related to a pancreatic cancer, the method comprising:
obtaining a histological sample of a pancreatic cancer tumor of a patient;
determining a feature set for the histological sample by applying a deep learning module trained on a population of histological samples of cancer tumors of the same type as the obtained histological sample of the cancer tumor; and
generating an outcome set for the patient by applying a second model to the determined feature set.

2. The method of claim 1, wherein the outcome set comprises at least one of a risk category, or risk-score for at least one of time to treatment discontinuation, time on treatment, recurrence free survival, progression free survival, event free survival, overall survival, response to therapy, or disease-free survival.

3. The method of claim 1, wherein the pancreatic cancer is at least one of unresectable pancreatic ductal adenocarcinoma, metastatic pancreatic ductal adenocarcinoma and resectable pancreatic ductal adenocarcinoma.

4. The method of claim 1, further comprising:
providing a set of recommended therapies responsive to the determined feature set for the histological sample.

5. The method of claim 1, wherein the feature set for the histological sample comprises at least one of morphology data, tissue region data, spatial relationship data, colocalization data, and hotspot data.

6. The method of claim 1, wherein the deep learning module comprises a U-Net model, wherein the U-Net model comprises a fully convolutional neural network having an encoder and decoder.

7. The method of claim 1, further comprising:
training the deep learning module on the population of histological samples of cancer tumors of the same type as the obtained histological sample of the cancer tumor to determine nuclei location and shape data.

8. The method of claim 1, wherein determining a feature set for the histological sample further comprises:
determining locations of tissue within the histological sample;
detecting positions of nuclei and cells of interest within the determined locations of tissue;
determining at least one of morphologic, geometric, and textural features for each of the detected nuclei and cells of interest; and
determining a spatial location feature for each of the detected nuclei and cells of interest.

9. The method of claim 1, wherein the second model comprises a multivariate model.

10. The method of claim 9, wherein the multivariate model comprises a Cox proportional hazards (CPH) model.

11. The method of claim 1, further comprising:
training the second model on non-histological data comprising at least one of medical images, clinical variables, genomics, and medical text.

12. The method of claim 1, further comprising:
training the second model to determine a signature, wherein the signature comprises the combination of histological features and weights.

13. The method of claim 1, further comprising:
administering to the patient the particular treatment type, responsive to the outcome set for a particular treatment type.

14. The method of claim 1, further comprising:
displaying, on a graphical user interface, at least a portion of the outcome set.

15. A non-transitory computer-readable medium storing instructions that, when executed on one or more processors, cause the one or more processors to:
obtain a histological sample of a pancreatic cancer tumor of a patient;
determine a feature set for the histological sample by applying a deep learning module trained on a population of histological samples of cancer tumors of the same type as the obtained histological sample of the cancer tumor; and
generate an outcome set for the patient by applying a second model to the determined feature set.

16. The non-transitory computer-readable medium of claim 15, wherein the instructions further include instructions that cause the one or more processors to:
display, on a graphical user interface, at least a portion of the outcome set.

17. The non-transitory computer-readable medium of claim 15, wherein the instructions further include instructions that cause the one or more processors to determine the feature set for the histological sample by determining locations of tissue within the histological sample, detecting positions of nuclei and cells of interest within the determined locations of tissue, determining at least one of morphologic, geometric, and textural features for each of the detected nuclei and cells of interest, or determining a spatial location feature for each of the detected nuclei and cells of interest.

18. The non-transitory computer-readable medium of claim 15, wherein the second model comprises a multivariate model.

19. A system for predicting outcomes related to a pancreatic cancer, the system comprising:
at least one server communicatively coupled to a user device by a network, wherein the at least one server further comprises a non-transitory memory storing computer-readable instructions and at least one processor;
the execution of the computer-readable instructions causing the at least one server to:
train a deep learning module on a population of histological samples of pancreatic cancer tumors, wherein the deep learning module comprises a U-net model;
train a second model on feature set data and outcomes data, wherein the second model comprises a multivariate model;
obtain a histological sample of a cancer tumor of a patient, wherein the cancer tumor is of the same type as the population of histological samples of cancer tumors;
determine a feature set for the histological sample by applying the trained deep learning module; and
generate an outcome set for the patient by applying the trained multivariate model to the determined feature set.

20. The system of claim 19, wherein the feature set comprises at least one of morphology data, tissue region data, spatial relationship data, colocalization data, and hotspot data.

21. The system of claim 19, wherein determining the feature set comprises the execution of computer-readable instructions causing the at least one server to:
determine locations of tissue within the histological sample;
detect positions of nuclei and cells of interest within the determined locations of tissue;
determine at least one of morphologic, geometric, and textural features for each of the detected nuclei and cells of interest; or
determine a spatial location feature for each of the detected nuclei and cells of interest.

22. The system of claim 19, wherein the outcome set comprises at least one of a risk category, or risk-score for at least one of time on treatment, time to treatment discontinuation, recurrence free survival, progression free survival, event free survival, overall survival, response to therapy, or disease-free survival.

23. The system of claim 19, further comprising a graphical user interface, communicatively coupled to the at least one server, wherein the graphical user interface is configured to display a portion of the outcome set.

24. A method for providing a set of recommended therapies related to pancreatic cancer, the method comprising:
obtaining a histological sample of a pancreatic cancer tumor of a patient;
determining a feature set for the histological sample by applying a deep learning module trained on a training data set, wherein the training data set comprises pancreatic cancer treatment outcomes for a prospective therapy;
generate an outcome set for the patient by applying a second model to the determined feature set;
determining a signature for the histological sample by thresholding the generated outcome set; and
providing a set of recommended therapies related to pancreatic cancer responsive to the determined signature.

25. The method of claim 24, wherein the prospective therapy comprises a combination therapy.

26. The method of claim 24, wherein the outcome set comprises at least one of a risk category, or risk-score for at least one of time to treatment discontinuation, time on treatment, recurrence free survival, progression free survival, event free survival, overall survival, response to therapy, or disease-free survival.

27. The method of claim 24, wherein the pancreatic cancer is at least one of unresectable pancreatic ductal adenocarcinoma, metastatic pancreatic ductal adenocarcinoma and resectable pancreatic ductal adenocarcinoma.

28. The method of claim 24, wherein the feature set for the histological sample comprises at least one of morphology data, tissue region data, spatial relationship data, colocalization data, and hotspot data.

29. The method of claim 24, wherein the deep learning module comprises a U-Net model, wherein the U-Net model comprises a fully convolutional neural network having an encoder and decoder.

30. The method of claim 24, further comprising:
training the deep learning module on the population of histological samples of cancer tumors of the same type as the obtained histological sample of the cancer tumor to determine nuclei location and shape data.

31. The method of claim 24, wherein determining a feature set for the histological sample further comprises:
determining locations of tissue within the histological sample;
detecting positions of nuclei and cells of interest within the determined locations of tissue;
determining at least one of morphologic, geometric, and textural features for each of the detected nuclei and cells of interest; and
determining a spatial location feature for each of the detected nuclei and cells of interest.

32. The method of claim 24, wherein the second model comprises a multivariate model.

33. The method of claim 32, wherein the multivariate model comprises a Cox proportional hazards (CPH) model.

34. The method of claim 24, further comprising:
training the second model on non-histological data comprising at least one of medical images, clinical variables, genomics, and medical text.

35. The method of claim 24, wherein the signature comprises the combination of histological features and weights.

36. The method of claim 24, further comprising:
administering to the patient the particular treatment type, responsive to the outcome set for a particular treatment type.

37. The method of claim 24, further comprising:
displaying, on a graphical user interface, at least a portion of the outcome set.
